(19) United States
(12) Patent Application Publication
(10) Pub. No.: US 2025/0229136 A1
(43) Pub. Date: Jul. 17, 2025

(54) AUTONOMIC NERVOUS SYSTEM REGULATION WITH PERSONALIZED TRIGGERS

(71) Applicant: MMNTSIPE, LLC, Houston, TX (US)

(72) Inventors: Ryan Boehner, Nacogdoches, TX (US); John Cannella, Mountain View, CA (US)

(21) Appl. No.: 19/017,275

(22) Filed: Jan. 10, 2025

Related U.S. Application Data
(60) Provisional application No. 63/620,693, filed on Jan. 12, 2024.

(51) Int. Cl. A63B 24/00; A61B 5/0245; A61B 5/352; A63B 71/06

(52) U.S. Cl. CPC A63B 24/0075; A61B 5/0245; A61B 5/352; A63B 24/0062; A63B 71/0622; A63B 2230/04

(57) ABSTRACT

A device performs an autonomic nervous system (ANS) regulation program for regulation of a user's ANS. The ANS regulation program may be tailored to the user based on biometric data of the user. The device initializes the program comprising a state model comprising a plurality of states, wherein each state includes one or more criteria for transitioning to another state of the plurality of states. The device obtains a heart rate signal, via health sensor(s) coupled to the client device during the program. For each state, the device prompts the user to perform one or more actions to regulate an ANS of the user. The device determines whether the heart rate signal obtained during the one or more actions satisfy the one or more criteria for the state. Responsive to determining that the one or more criteria are satisfied, the device transitions to another state of the state model.

### CROSS-REFERENCE TO RELATED APPLICATION

This application claims the benefit of and priority to U.S. Patent Application No. 63/620,693, filed Jan. 12, 2024, the contents of which are incorporated by reference in its entirety.

### BACKGROUND

There currently exist HIIT (“high intensity interval training”) and SIT (“sprint intensity training”) programs. These modalities of physical exercise may be as effective or more effective than MICT (“moderate intensity continuous training”) programs for general health improvements. Other programs include breathing techniques, including hyperventilation and self-induced apnea or hypoxia. Similarly, Valsava maneuvers are a breathing technique used by power lifting athletes and personal trainers to stabilize the core. However, many of these modalities fail to take into account each individual's response to the various programs or actions. While one activity could be extremely stressing to one individual, it could, on the other hand, be a fairly mild stressor to another. There is a need for custom tailoring of programs to suit each user's physiological state and fitness needs.

### SUMMARY

According to one or more aspects, the techniques described herein relate to a computer-implemented method including: initializing, via a client device, an autonomic nervous system (ANS) regulation program including a state model including a plurality of states, wherein each state includes one or more criteria for transitioning to another state of the plurality of states; obtaining a heart rate signal, via one or more health sensors wirelessly coupled to the client device during the ANS regulation program; and for each state of the state model: prompting, via the client device, a user of the client device to perform one or more actions to regulate the ANS of the user, determining whether the heart rate signal obtained during the one or more actions for the state satisfy the one or more criteria for the state, and responsive to determining that the one or more criteria are satisfied, transitioning to another state of the plurality of states.

In some aspects, the techniques described herein relate to a computer-implemented method including: initializing, via a client device, an autonomic nervous system (ANS) regulation program including a state model including a plurality of states, wherein each state includes one or more criteria for transitioning to another state of the plurality of states; obtaining a heart rate signal, via one or more health sensors wirelessly coupled to the client device during the ANS regulation program; and for each state of the state model: prompting, via the client device, a user of the client device to perform one or more actions to regulate an ANS of the user, determining whether the heart rate signal obtained during the one or more actions for the state satisfy the one or more criteria for the state, and responsive to determining that the one or more criteria are satisfied, transitioning to another state of the plurality of states.

In some aspects, the techniques described herein relate to a computer-implemented method, wherein initializing the ANS regulation program includes presenting, via the client device, a user interface on an electronic display of the client device, wherein prompting the user to perform the one or more actions in each state includes prompting the user via one or more graphical elements in the user interface presented on the electronic display of the client device.

In some aspects, the techniques described herein relate to a computer-implemented method, wherein determining whether the heart rate signal obtained during the one or more actions for the state satisfy the one or more criteria for the state includes: determining whether the heart rate signal reaches a target heart rate for the one or more actions; and prompting, via the client device, the user to end the one or more actions.

In some aspects, the techniques described herein relate to a computer-implemented method, wherein determining whether the heart rate signal obtained during the one or more actions for the state satisfy the one or more criteria for the state includes: determining whether the heart rate signal reaches a recovery level, following prompting of the user to end the one or more actions.

In some aspects, the techniques described herein relate to a computer-implemented method, wherein obtaining the heart rate signal includes one or both of: obtaining the heart rate signal from a heart rate monitor coupled to the user; and calculating the heart rate signal from R-R intervals of an electrocardiogram signal obtained from a electrocardiogram monitor.

In some aspects, the techniques described herein relate to a computer-implemented method, further including: for an initial state of the state model, determining whether a physiological state of the user is stable based on the heart rate signal.

In some aspects, the techniques described herein relate to a computer-implemented method, wherein determining whether the physiological state of the user is stable includes: calculating a slope of the heart rate signal over a past window of time; and determining whether the slope of the heart rate signal is within a tolerance of a zero slope.

In some aspects, the techniques described herein relate to a computer-implemented method, wherein determining whether the physiological state of the user is stable includes: calculating a spread of the heart rate signal over a past window of time as a difference between a maximum value and a minimum value in the past window of time; and determining whether the spread of the heart rate signal is within a threshold spread.

In some aspects, the techniques described herein relate to a computer-implemented method, further including: for at least one state: identifying a frequency in oscillation of the heart rate signal; and determining a start time of an exercise based on the frequency in the oscillation of the heart rate signal to align the exercise with an incline in the heart rate signal.

In some aspects, the techniques described herein relate to a computer-implemented method, further including: for each state of the state model: responsive to determining that the one or more criteria are not satisfied, continuing the state.

In some aspects, the techniques described herein relate to a non-transitory computer-readable storage medium storing instructions that, when executed by a computer processor, cause the computer processor to perform operations including: initializing, via a client device, an autonomic nervous system (ANS) regulation program including a state model including a plurality of states, wherein each state includes one or more criteria for transitioning to another state of the plurality of states; obtaining a heart rate signal, via one or more health sensors wirelessly coupled to the client device during the ANS regulation program; and for each state of the state model: prompting, via the client device, a user of the client device to perform one or more actions to regulate an ANS of the user, determining whether the heart rate signal obtained during the one or more actions for the state satisfy the one or more criteria for the state, and responsive to determining that the one or more criteria are satisfied, transitioning to another state of the plurality of states.

In some aspects, the techniques described herein relate to a non-transitory computer-readable storage medium, wherein initializing the ANS regulation program includes presenting, via the client device, a user interface on an electronic display of the client device, and wherein prompting the user to perform the one or more actions in each state includes prompting the user via one or more graphical elements in the user interface presented on the electronic display of the client device.

In some aspects, the techniques described herein relate to a non-transitory computer-readable storage medium, wherein determining whether the heart rate signal obtained during the one or more actions for the state satisfy the one or more criteria for the state includes: determining whether the heart rate signal reaches a target heart rate for the one or more actions; and prompting, via the client device, the user to end the one or more actions.

In some aspects, the techniques described herein relate to a non-transitory computer-readable storage medium, wherein determining whether the heart rate signal obtained during the one or more actions for the state satisfy the one or more criteria for the state includes: determining whether the heart rate signal reaches a recovery level, following prompting of the user to end the one or more actions.

In some aspects, the techniques described herein relate to a non-transitory computer-readable storage medium, wherein obtaining the heart rate signal includes one or both of: obtaining the heart rate signal from a heart rate monitor coupled to the user; and calculating the heart rate signal from R-R intervals of an electrocardiogram signal obtained from a electrocardiogram monitor.

In some aspects, the techniques described herein relate to a non-transitory computer-readable storage medium, the operations further including: for an initial state of the state model, determining whether a physiological state of the user is stable based on the heart rate signal.

In some aspects, the techniques described herein relate to a non-transitory computer-readable storage medium, wherein determining whether the physiological state of the user is stable includes: calculating a slope of the heart rate signal over a past window of time; and determining whether the slope of the heart rate signal is within a tolerance of a zero slope.

In some aspects, the techniques described herein relate to a non-transitory computer-readable storage medium, wherein determining whether the physiological state of the user is stable includes: calculating a spread of the heart rate signal over a past window of time as a difference between a maximum value and a minimum value in the past window of time; and determining whether the spread of the heart rate signal is within a threshold spread.

In some aspects, the techniques described herein relate to a non-transitory computer-readable storage medium, the operations further including: for at least one state: identifying a frequency in oscillation of the heart rate signal; and determining a start time of an exercise based on the frequency in the oscillation of the heart rate signal to align the exercise with an incline in the heart rate signal.

In some aspects, the techniques described herein relate to a system including: a computer processor; and a non-transitory computer-readable storage medium storing instructions that, when executed by the computer processor, cause the computer processor to perform operations including: initializing, via a client device, an autonomic nervous system (ANS) regulation program including a state model including a plurality of states, wherein each state includes one or more criteria for transitioning to another state of the plurality of states; obtaining a heart rate signal, via one or more health sensors wirelessly coupled to the client device during the ANS regulation program; and for each state of the state model: prompting, via the client device, a user of the client device to perform one or more actions to regulate an ANS of the user, determining whether the heart rate signal obtained during the one or more actions for the state satisfy the one or more criteria for the state, and responsive to determining that the one or more criteria are satisfied, transitioning to another state of the plurality of states.

### BRIEF DESCRIPTION OF THE DRAWINGS

Figure (FIG.) 1 illustrates a networking environment of an ANS regulation system, according to one or more embodiments.

FIG. 2 is a block diagram illustrating an architecture of an ANS regulation system, according to one or more embodiments.

FIG. 3 illustrates an example unit in a state model, according to one or more embodiments.

FIG. 4 illustrates identifying an HR stabilized point for an initial state of an ANS regulation program, according to one or more embodiments.

FIG. 5 illustrates identifying the HR stabilized point for example data of a user, during an initial state of an ANS regulation program, according to one or more example implementations.

FIG. 6 illustrates identifying a stressor start time during an ANS regulation program, according to one or more embodiments.

FIG. 7 illustrates identifying a stressor start time during an ANS regulation program, according to one or more example implementations.

FIG. 8 illustrates identifying a recovery start time during an ANS regulation program, according to one or more embodiments.

FIG. 9 illustrates identifying a recovery start time during an ANS regulation program, according to one or more example implementations.

FIG. 10 illustrates identifying a recovery start time for a respiratory-based stressor during an ANS regulation program, according to one or more embodiments.

FIG. 11 illustrates identifying a recovery start time for a respiratory-based stressor during an ANS regulation program, according to one or more example implementations.

FIG. 12 illustrates identifying a full recovery following a stressor during an ANS regulation program, according to one or more embodiments.

FIG. 13 illustrates identifying a full recovery following a stressor during an ANS regulation program, according to one or more example implementations.

FIG. 14 illustrates a process of generating a tailored ANS regulation program, according to one or more embodiments.

FIG. 15 illustrates a process of provision of an ANS regulation program, according to one or more embodiments.

### DETAILED DESCRIPTION

The Figures (FIGS.) and the following description relate to preferred embodiments by way of illustration only. It should be noted that from the following discussion, alternative embodiments of the structures and methods disclosed herein will be readily recognized as viable alternatives that may be employed without departing from the principles of what is claimed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

### Overview of the Autonomic Nervous System

Clinical programs like Heart Rate Variability HRV-Biofeedback (“HRV-BFB”) train users to remain calm throughout stressful situations. Generally, increased Heart Rate Variability (“HRV”) may be positively correlated with a range of positive health outcomes, from a reduction in all-cause mortality, to increase in behavioral flexibility. Increased HRV (with rare pathological exceptions) reflects improved relaxation/parasympathetic shift. Meditation, slow breathing with HRV-BFB, and yoga can all improve HRV. Through currently available methods, improved HRV is typically slight (on the order of 0-6 ms in industry-standard RMSSD (“Root Mean Square of Successive Difference”) scores), temporary (sometimes only measurable immediately after a session), and in some cases only visible in their absence (e.g., meditation or HRV-BFB can help a user's HRV avoid dips during stressful periods).

Further, it is known that chronic stress produces toxic by-products, both biological and psychological. Shorter bursts of stress, also sometimes described as acute stressors or as part of hormesis, may be more survivable. This is consistent with our understanding and application of acute stressors. Chronic toxicity relates to the idea that our bodies' and minds' responses to stresses become self-toxic when the stressors (or our residual responses to stressors) overwhelm the body's ability to respond. This can develop very quickly, depending on if the intensity or type or frequency of stress lasts too long, or when users experience more intense stressors than tolerable, or when users experience too many stressors without adequate recovery.

There is a need for programs to obtain diagnostic biometric data from a user, and use that data to produce scalable, mass-customizable, long-term, condition- and history-specific results, for example, a program that may increase both health and lifespan, stress tolerance and emotional flexibility.

Aspects of the embodiments herein provide constructive and destructive interference algorithmically. In a free-scale, real time way, methods herein shape an individual's chronobiology. While many currently see the Autonomic Nervous System (“ANS”) as steady state, programs disclosed herein are inherently nonlinear and oscillatory. Typical users activate their parasympathetic state by training with relaxation/meditation, but users can more deeply and effectively activate their parasympathetic systems by producing a rebound effect from acute stress moments. In typical reference, the sympathetic response is described as “bad” and the parasympathetic response as “good.” However, in the embodiments described herein, both responses are referred to as helpful and healthy responses.

ANS may respond to, and can be reprogrammed by, many forms of stressors, including light, exertion, respiration, temperature, sleep, emotion, and nutrition. Pattern recognition and similar repatterning—sessions of acute stressors—can apply across multiple types of stressors, or even apply multiple stressors simultaneously. Different levers can affect different symptoms, but all rewire the ANS. ANS dysregulation—and failure to remain in deep-enough parasympathetic activation—contributes to many chronic illnesses, including aging. By allowing the body to oscillate appropriately between rare acute varied stresses and longer, deep parasympathetic recovery, the body can shift into self-repair mode. Appropriate stress response is important. Human beings may benefit from useful and necessary adaptations. Programs disclosed herein train the body, mind, and brain to produce responses proportionate to the stress in the moment, and to recover quickly as the stress passes.

The regulation system may apply constructive interference, charting, PID (“Proportional Integral Derivative”) analog controller understanding, fractal mathematics, novel machine learning techniques, or some combination thereof to analyze users' HR and HRV. The systems and methods may apply artificial intelligence (or more specifically machine learning) to recognize how different stressors (e.g., exertion vs apneic exertion vs emotion and exertion) produce different-looking signals, and use that learning to modify real-time recommendations.

Without being bound to a particular underlying scientific basis, it appears much of aging and chronic disease finds its roots in ANS dysfunction and can be treated by improving that core system's ability to respond to and recover from stressors of different types. Heart rate (“HR”) and heart rate variability (“HRV”) may serve as initial proxies for ANS state and functioning; HRV is inversely correlated with all-cause mortality. HRV may be used as a primary independent variable of longevity. Biometric data (subsets of which are called Aggregated Stress Data (“ASD”) elsewhere in this description) refers to a suite of data that may include but is not limited to HR, HRV, temperature, SpO2 (“Saturation of Peripheral Oxygen”), accelerometer, location data, C-Reactive Protein (a measure of inflammation), telomere lengths, hormone panels, etc.

A program may use industry-standard inexpensive hardware and produce positive outcomes measurable using industry standard calculations. A technical analysis (similar to charting in financial markets) and signal processing techniques may be used to identify novel patterns in the data, and can then customize the user's recommendations based on the patterns identified and the patterns to be created. Certain patterns may produce better health outcomes or address specific health concerns. Technical analysis involves error correcting the data from multiple sources, correcting scale so the sources are comparable, adjusting for metrics measured (e.g., apnea looks very different when measured by a heart rate monitor than when measured by a pulse oximeter), so that specific signals may be detected. Signals may be categorized and drawn. Signals, including specific ratios and data in these signals, may be used as part of user profiling, as part of diagnostic methods, to identify specific therapeutic methods, and to identify timing for application of specific therapeutic methods.

Seven different levers—light, exertion, respiration, temperature, emotion, sleep, and nutrition—may act as effective types or subtypes in re-regulating the ANS. Levers may be used individually and multiple simultaneous levers may be used. Variations of stressors may also be used (e.g., apnea, serial apnea, different types of emotional response, etc.) for additional granularity in data analysis. Levers may be applied in specific ways, at specific times, for specific durations, and in specific intensities to assist the body in oscillating its sympathetic and parasympathetic system activations. Sessions of stressors may be applied at specific times of the day, week, and month to embed them in, help reestablish, and help reinforce the body's natural decadal, annual, lunar, weekly, daily, ultradian (i.e., intraday), and ECG (“electrocardiogram”) rhythms. Stressors may be applied in a specific pattern of diverging oscillation: each moment of acute stress followed by recovery (often governed by specific breathing patterns), followed by more moments of acute stress. Each moment produces a stronger stress response than the last. Calculations prescribe the timing, duration, intensity, and types of stressors applied. The program may combine stressors or use levers in different ways to recover before proceeding to apply additional stressors. The program stops at a point of desired local maximum acute stress and ends with meditative recovery of prescribed length and depth. Reprogramming and repatterning of the ANS may happen during a meditative recovery period.

The systems, methods, and/or computer readable medium storing program code (e.g., configuration and/or configurations) disclosed herein gather, analyze, and produce recommendations from time series information on stress levels, at least partially in real time, from users. The analysis and recommendations will specifically seek to improve autonomic nervous system functioning and metrics associated with lifespan and health. In some embodiments, the algorithm processes time series information, using this data as a proxy for stress or recovery responses in the user. In some embodiments, algorithms may use human-designed formulae, machine-learning-derived correlations, user-input (both spontaneous or prompted by the program), or a combination of the above to produce recommendations. The data can be gathered through a variety of devices, both proprietary and available broadly to consumers. Examples might include, but are not limited to, optical sensors worn on arms or wrists or chests or elsewhere, electrode heart rate monitor, electrocardiogram monitor, microphones placed in glasses or worn elsewhere, temperature sensors and accelerometers included in a variety of phones and wearable devices, embedded into clothing, and information gathered by input or feedback from the user.

Examples of the data collected include respiration, accelerometer, audio recording, heart rate or ECG, oxygenation (SpO2) readings, temperature (both ambient and body), UV exposure, and electrodermal data. These streams of data are Aggregated Stress Data (“ASD”). In some embodiments, a configuration may gather ambient information (e.g., location, weather, and altitude) data that informs the analysis of how the user responds to stress. In some embodiments, the configuration may include gathering user-specific information like age, sex, and personal history, which can assist in the user profiling part of the analysis. The algorithm may process ASD on a combination of a local device (e.g., desktop, laptop, mobile, wearable, etc.), in the cloud, or on remote servers, and returns observations and recommendations to the user. The recommendations may seek to address specific concerns and improve long-term health outcomes. The algorithm attempts to gather as much information as possible over the longest period of time available. This may include data collected for the user upon waking, before bed, throughout their day, and during activity windows such as workouts. For example, biometric readings taken upon waking and during prescribed activity windows may improve precision, analysis, and recommendations by including data from multiple sources over the span of each day. The program uses an immediately available rule set, either on a local device or in the cloud, to analyze and provide recommendations to the user.

The figures (FIGS.) illustrate example methods, design flows, and systems for programs to obtain diagnostic biometric data from a user and use that to produce scalable, mass-customizable, long-term, condition- and history-specific results, for example, a program that may produce HRV increases that are much more significant in magnitude and longer-lasting.

In illustrations, unless otherwise labeled, the x axis represents time; the y axis represents magnitude or positive scoring. In most cases, unless otherwise noted as representing a specific stressor, graphs represent either heart rate over time or a generic visualization of real time stress-response data.

### Networking Environment of ANS Reregulation System

FIG. 1 illustrates a networking environment of an ANS regulation system 120, according to one or more embodiments. The networking environment includes at least a client device 100, a health sensor 110, and the ANS regulation system 120. In other embodiments, the networking environment may include additional components, e.g., a third-party system 130, a network 140, other computing devices, etc. In other embodiments, there may be multiple of each component, e.g., multiple health sensors 110, multiple client devices 100, etc.

The client device 100 provides biometric data 115 to the ANS regulation system 120. The client device 100 may be communicatively coupled to the health sensor 110. The client device 100 is a computing device in use by a user. The client device 100 may include, among other computing components, a processor and a computer-readable storage medium storing instructions to be executed by the processor. The client device 100 may also include an electronic display, or other output devices for presenting information to the user. The electronic display, for example, may be used to provide a graphical user interface for the user. Through the graphical user interface, the client device 100 may present an ANS regulation program for regulating the user's ANS system. The user interface may also be used to receive user input from the user. Another output device may include an audio speaker system configured to present audio content to the user. In one or more embodiments, the client device 100 receives biometric data from the health sensor 110 and transmits the biometric data to the ANS regulation system 120 for analysis. The ANS regulation system 120 may provide instructions for performing the ANS regulation program 125 to the client device 100, e.g., tailored based on the biometric data 115. In one or more embodiments, the client device 100 may execute an application for communication with the ANS regulation system 120. In other embodiments, the ANS regulation system 120 may provide the ANS regulation program 125 to the client device 100, with which the client device 100 may analyze the biometric data 115 and perform the ANS regulation program 125. In one or more embodiments, the client device 100 may process the biometric data 115, prior to transmission to the ANS regulation system 120. For example, processing may entail correcting errors or artifacts in the biometric data, assessing data quality, re-formatting data, compressing data, etc.

The health sensor 110 gathers biometric data from a user of the client device 100. The health sensor 110 is configured to record biometric data of the user. Example biometric data, collectable by the health sensor 110, includes cardiovascular data such as an electrocardiogram (ECG), heart rate, and heart rate variability; respiratory data such as respiratory rate and blood oxygen saturation; movement and activity data such as step count, distance traveled, calories burned, and sleep tracking; and other physiological data such as body temperature, electrodermal activity (EDA), galvanic skin response (GSR), and electromyography (EMG). This diverse data collection capability enables the health sensor to provide comprehensive insights into an individual's current physiological state. The health sensor 110 may be configured as a wearable device, such as a wristband, armband, or chest strap. The health sensor 110 may be configured for continuous or intermittent monitoring of the individual's physiological state. In some embodiments, the health sensor 110 may be integrated as a component of the client device 100 mobile device, such as a smartphone or smartwatch, providing a convenient and readily accessible platform for health monitoring.

The client device 100 performs the ANS regulation program 125 to regulate the user's ANS system. In performing the ANS regulation program 125, the client device 100 presents one or more prompts to the user, e.g., via one or more output devices, to stress the user's ANS system. The client device 100 may further present one or more prompts to the user, e.g., via one or more output devices, to recover the user's ANS system. The client device 100 may obtain biometric data 115 from the user, e.g., via the health sensor 110, during performance of the ANS regulation program 125. The client device 100 may provide the biometric data 115 to the ANS regulation system 120 for analysis, or may perform analyses on the biometric data 115 to inform progression of the ANS regulation program 125.

In one or more embodiments, the client device 100 may provide other recommendations to the user, e.g., for regulation of the user's ANS system. The client device 100 may provide such recommendations via visible communications (e.g., visual content displayed on a smartphone screen or laptop screen), auditory communications (e.g., audio content presented by a smartphone speaker or by speakers on a smart eyeglass set), holographic communications (e.g., using either a projector or projected onto augmented reality equipment), tactile communications (e.g., communicated via haptic clothing, or vibration in phone or smartwatch), or some combination thereof. Recommendations may include real-time interventions, longer-term planning, or some combination thereof. An example of short-term, real-time intervention might include an observation of lower heart rate variability and higher heart rate—both indicative of sympathetic nervous system activation—during a morning reading. The system might inquire if the user feels they might be ill or might be able to identify the source of the stress. If the user feels they might have been exposed to a possible illness, the system might suggest skipping that day's scheduled session (or recommendations) and instead engaging in different actions or recommendations, e.g., temperature modulation sessions at prescribed intervals to marshal the immune system. An example of longer-term planning may include a calendar function.

In some embodiments, the ANS regulation system 120 collects the biometric data and other user data from the client device 110 for analysis and generation of recommendations. In one or more embodiments, the ANS regulation system 120 may be integrated with the user's calendar software, whereby the user may be assisted to identify optimal “activity windows.” The term “activity windows” describes periods when the user might best benefit from periods of stress and activity, especially “sessions” of acute stressors as prescribed by the program. Optimal periods may be identified for fasting (sometimes as specific as which meals to skip), nutrient-cycling, and nutrient-timing, and assist the user to plan their calendar around these recommendations.

The third-party system 130 may store other data usable in analyses for regulation of the ANS system. The third-party system 130 may be a computing device configured to perform computer functionality. The third-party system 130 may store data usable in the analyses for regulation of the ANS system. In one or more embodiments, the third-party system 130 may be an electronics health record database, storing health data for one or more individuals. For example, with permission from the individual, the third-party system 130 may provide health data of the individual to the client device 100 and/or the ANS regulation system 120. The ANS regulation system 120 may leverage the health data in performing analyses for regulation of the ANS system of the individual. In other embodiments, the third-party system 130 stores data on studies linking ANS system regulation with treatment of various health issues. The ANS regulation system 120 may obtain such data to generate and/or modify one or more ANS regulation programs to target treatment of the various health issues based on the data from the studies. In other embodiments, the third-party system 130 stores health data on a population of users. The ANS regulation system 120 may leverage such data to determine the various stressors and actions in an ANS regulation program, e.g., informed by responses to stressors across the population of users.

The network 140 connects devices together for communication of data between the devices. The network 140 may be any suitable communications network for data transmission, e.g., any wireless or wired connectivity protocol. In one or more embodiments, the network 140 uses standard communications technologies and/or protocols and can include the Internet. In one or more examples, the network 140 may leverage ethernet, fiber optic, universal serial bus, thunderbolt, high-definition multimedia interface, Wi-Fi, Bluetooth, cellular, Zigbee, wide area network, local area network, other connectivity protocols, or some combination thereof. In another embodiment, the network 140 use custom and/or dedicated data communications technologies. In one or more embodiments, functionality of the various devices may be performed locally on the device, or performed via cloud computing, empowered by the network 140.

### Example Health Applications for Ans Regulation

The configurations disclosed herein include programs that may produce meaningful upward shifts in morning resting Heart Rate Variability (HRV) readings and meaningful parasympathetic shifts and improvements in functioning in the Autonomic Nervous System (ANS). HRV is a strong correlate with longevity, and a strong inverse correlation with cognitive decline and chronic illness. Programs disclosed herein may produce significant and long-lasting HRV increases on the order of 15-150 milliseconds (ms) in industry-standard RMSSD scores, based on program adherence. The various recommendations may be scalable/mass-customizable, long-term, condition- and history-specific results. For example, the ANS regulation system 120 may quantify and integrate annual, seasonal, monthly, daily, and/or ultradian rhythms into personalized recommendations including specific diagnostic and therapeutic signals.

A protocol may involve a combination of overall longer-term healthy behaviors and applying one or some combination of acute stressors in “moments.” A given prescribed group of “moments” is called a “session.” A session is a period of prescribed punctuated stress and recovery. Moments describes individual acute periods of stress application within a program. Compound moments are when more than one stressor during any one moment is applied, for example, apnea plus exertion, or temperature plus exertion. Acute moments are moments of 4-60 seconds in duration.

Within the first two hours upon waking each day, the user takes a morning baseline reading. The reading provides both an absolute-value indicator for the user's health, and a relative value, relative to both the user's prior baseline, and relative to peer cohort. An activity window may be assigned, defined as an optimal time of day to hold a session, or suggest recovery (or a day off). Activity windows may be forecast in advance. The user may be assisted in coordinating their meal timing and volumes with their activity windows and fasting needs. The program may sometimes schedule heavy meals, medium meals, lighter meals, or skipping meals. Meal timing relates to regularly scheduled meals. For example, a program may recommend a user eat a meal containing 20-30 grams of protein 15-60 minutes after each exertion or exertion compound session. A schedule may include: breakfast between 7:30 am-8:30 am, lunch between 12 noon-1:30 pm, and dinner between 6:00-7:30 pm. The program may also suggest different nutrient composition, coordinated with other aspects of the program. The program may use levers to apply stressors and train recovery, including, for example: exertion, respiration, temperature, emotion, sleep, light, and nutrition.

Exertion sessions may involve combinations of breath-holds and interval actions of increasing intensity. The specific timing for the breath-holds and intervals may be prescribed in real-time by the software. Based on biometric data collected in real-time and past information collected about users and that particular user, the program may also alter the session's length or composition. The program may include a punctuation breath, which is an inhalation hold used to 1) achieve more complete recovery and 2) produce small Pavlovian stress response in between acute moments. A punctuation breath begins when level local recovery is achieved and an upside signal is triggered. User inhales as deeply as possible and holds the inhalation. The program signals exhalation at the moment user's rolling HR begins to decline after second local maximum; user then exhales lungs as fully as possible and begins breathing normally.

Modified RSA (“Respiratory Sinus Arrhythmia”) breathing is a breathing pattern that attempts to establish and strengthen the user's RSA. This breathing pattern consists of a short 1-1.5 second inhale, a short 1-1.5 second exhale, and a 3-4.5 second hold on the exhales. It should feel mildly uncomfortable. Done in 3-5 minute sessions, it trains the user to slow pace of breaths per minute, reduce the total volume breathed and tidal volume breathed. The goal is to assist users with absent- or inverse-RSA to develop healthy positive RSA. PSA breathing is a breathing pattern that attempts to improve and deepen “Parasympathetic System Activation.” This consists of breathing with a roughly 4:6 ratio of inhale:exhale. Each inhalation-exhalation-hold cycle will typically last 10-12 seconds, although this will vary user by user. During the exhale, the user may be prompted to make a closed-mouth low-humming sound of decreasing tone. Overbreathing is breathing that is typically consciously prompted, by mouth, more rapid, and/or deeper than necessary at physical rest. Sometimes prescribed from breathwork practitioners to produce stress or holotropic reactions. Priming is breathing for short inhalation-hold periods, punctuated with normal breathing, that may be used before apneic moments. Nasal breathing is used to encourage slow nasal breathing with limited tidal volume the vast majority of the time, except for specific breathing actions or moments of acute exertion. Nasal breathing has the effect of producing a subtle temporary parasympathetic shift. Done regularly over the long term, however, this parasympathetic shift becomes a habit and can improve health. Panting breathing is rapid mouth breathing produced during and immediately following acute exertion and/or apnea moments. This is often accompanied by specific HR signals that may be analyzed and incorporate into real-time recommendations. Valsalva maneuver is a breathing maneuver where the user pushes the tongue up towards the roof of the mouth while exhaling, producing an increase in intrabdominal pressure and some tensing of the core. It increases heart rate and momentary slope when performed during muscular contraction portions of exertion or exertion compound moments. Hypoxia hypoventilation exhalation holds include self-induced moments of exhalation-holds, up to several in series, the combination up to 1 minute in duration. Such holds may be used to blunt hyperreactivity and psychological hypomania, or used to produce slingshot effect to increase peak escalation.

HR hyporeactivity may be observed as correlated to a “flat affect” in psychotherapeutic environments. HR hyporeactivity may be observed in endurance athletes as part of “heart rate decoupling.” However, these aspects are not currently seen as a negative outcome requiring change, and are not necessarily seen as correlated. This may be because the flatter affect does not produce the obvious short-term physical symptoms or psychological “acting out” observed in more hyperreactive people. Appropriately-reactive and hyporeactive people may be phenotypically similar, so they appear outwardly similar and normal. Heart Rate Variability Biofeedback Training (HRV-BFB) is a clinical practice in which the user controls his breathing in order to increase HRV in real time. In practice, the effect may be temporary and slight, compared to the meaningful long-term shift that may be possible through the program. Flattening is where modem society rewards “affect regulation,” flat vocal intonation and volume modulation, long steady exercise periods. This means many users have trained themselves and their heart rates over time to become emotionally and physically hyporeactive. Contamination is what happens during endurance actions, or MICT, that might affect and substantially diminish the positive effect of the program.

Diagnosis may include an analysis of stress loads. For example, if the resting morning HRV is low, but the user finds it easy to produce high heart rates during acute moments, hyperreactivity may be a possibility. Psychological questionnaires may be used to determine if HR seems decoupled from the stress load to determine if hyporeactivity is a possibility. Applying specific stressors of known magnitude, known to produce a given magnitude stress response in a subgroup of similar user profile characteristics; if the user substantially exceeds the typically stress response manifested in response to stress applied, hyper reactivity is a possible diagnosis; if user manifests substantially lower stress response than typical for a given applied stress, hyporeactivity may be a possible diagnosis. If a user or user's friends report a flat affect but not depression, then hyporeactivity may be a possibility. If users report disproportionately large emotional response to moderate perceived threats, hyperreactivity may be a possibility. sets in central data storage 120 may be used to categorize patient responses as hyporeactive, hyperreactive, appropriate-reactivity, or a gradation of each category.

Categorization may be as hyporeactive, appropriately-reactive, or hyperreactive. Categorization may also include scoring on a spectrum. A therapeutic method may be used to determine if hyperreactive, via emotional modulation/containment, or via more apnea/hypoxia, or via exertion compound moments building from backs of sessions. Based on the user's categorization, the system modifies a standard program to address the categorization.

For example, a program may establish deeper baselines through PSA breathing and longer meditative periods at ends of sessions; modify session targets to focus on hitting appropriate goals, not overreaching maximum heart rate; increase importance weighting of downward slope; serve as adjunctive therapy to Dialectrical Behavior Therapy (“DBT”) or suggest DBT as adjunctive therapy for this protocol. Therapeutic methods may include, if hyporeactive, in addition to standard protocol some combination of: add emotion modulation/breath sessions; add warm temperatures in general and especially during the beginning of stress moments; add deep inhalations before starting stressor moments; shorten time between punctuation breath and applying next stress moment during a stressor session; emphasize Valsalva maneuver; add more apnea/hypoxia plus exertion compound moments building from fronts of sessions, only after substantial progress using other stressors; add additional fasting periods; or focus sessions more on stretching to max heart rate per moment.

A shallow recovery slope program may relate to systems and methods for diagnosing and quantifying rate and depth of recovery from initial stress responses, and for promoting appropriately-rapid and organized recovery. Methods for establishing a diagnosis may include verifying normal slope (as seen in user subgroups with similar characteristics) vs shallow slope in this particular user, and gradations thereof. A shallow slope may imply slow recovery from an acute stress moment or reduced capacity as described. Once diagnosed, therapeutic methods may include in addition to standard protocol: emphasis on brief stress moments; emphasis on stillness during acute recovery; excise other chronic stressors and endurance exercise; identify chemical contributors (e.g., caffeine) and recommend their gradual cessation; audit sleep hygiene including blue light exposure; and audit possibility of over-eating.

Recovery slope is the rate at which average rolling ASD decreases following cessation of stressor. Recovery depth is short-term, the new relative baseline and parasympathetic shift compared to the baseline immediately before the prior moment. In the long-term, recovery slope can describe a meaningful parasympathetic shift of the ANS in users during their everyday lives. Not-filling-the-void: once a user establishes a deep parasympathetic state, the user's own rhythms will begin to rebel against that state. Many users may be addicted to patterns of chaos and disorganization. Disorganization refers to how long wavelength, low fractality (meaning little variation) stressors, especially those applied at the “wrong” times of the day, may produce disorganization and illness, resulting in hyporeactivity or hyperreactivity. Part of a program may involve coaching the individual user to anticipate and experience the anxiety and temptations-to-chaos as temporary. This can be seen as a cousin of Peter Levine's somatic experiencing or Dan Siegel's mindsight work, within the oscillatory and deeper shifts the programs enable.

A lack of increasing baseline program may relate to systems, methods, and computer readable mediums that provide programs for diagnosing and quantifying how the autonomic nervous system responds to repeated acute stresses and produces a cumulative stress response, and for promoting appropriate underlying cumulative response. A therapeutic method may include adequate deep rest, sleep hygiene, extra sun exposure, or warm temperatures during acute stressor sessions.

Another aspect may relate to systems and methods promoting reduction in Gastroesophageal Reflux Disease (GERD) symptoms, reducing body weight or Body Mass Index (BMI), or reducing symptoms or complications related to obesity. In addition to standard diagnostic criteria (e.g., doctor diagnoses), the application may add additional insight to explore or confirm diagnoses. For example, the application may be able to find correlations between late or large meals and sympathetic nervous system (SNS) activation in real-time, between SNS activation and poor sleep, and between poor sleep and low HRV scores the next morning. Typically, eating food serves as recovery, providing energy and nutrients. One insight of the algorithm is that over-eating food “flips the switch,” and that beyond a certain point, the body becomes sympathetically activated by the food. This can occur because of quantity consumed, eating too quickly, or quality (e.g, high glycemic index) of the food consumed. This means the algorithm may attempt to find signs of this switch in the user's ASD, indicating a need for a modified program to assist the user in controlling their SNS. Therapeutic methods applied to users with this particular set of goals or issues may include: Periodic Coordinated Fasting (PCF); RSA, PSA, or other meditative breathing for 15-20 minutes immediately preceding meals; eating heavier breakfasts and lunches with lighter dinners; specific focus on ANS listening; modified software prompts (e.g., drinking water, somatic experiencing of fullness, noting if the user's appears sympathetically activated before meals, or noting if the person's ASD indicates over-eating in real-time); or catabolic/nutrient cycling.

A conflicting ultradian rhythms program may relate to systems and methods for diagnosing and quantifying how the autonomic nervous system develops chaotic or conflicting intraday rhythms, and for promoting appropriately coherent and distinct intraday rhythms. Human beings experience many forms of intraday rhythms, such as the energy lull and sugar- or caffeine-cravings that people report between 1 μm and 3 pm. Intraday rhythms are typically described as “ultradian.” In sleep, ultradian rhythms appear most often to manifest as 90-120 minute cycling of sleep stages. Chronobiologically-healthier users experience noticeable 90-120 minute cycling of energy levels throughout the day, although some healthier users may experience cycles of as long as 4 hours in some aspects of their lives (e.g., their Dopaminergic system). Many chronic stressors of modern life (e.g., caffeine, alcohol, overeating, long periods of video game playing, and endurance exercise) can elongate or disrupt these rhythms. These disrupted, elongated, chaotic, or conflicting ultradian rhythms may be diagnosed via techniques analyzing the ASD. This diagnostic method adapted the standard protocol to include continuous monitoring for several days and prompting at key moments throughout the monitoring period. The program may include an interview, delivered either by a human being or via automated questionnaire, to confirm the diagnosis.

Just as ultradian rhythms can be stretched/elongated, they can be shortened. It may not always benefit the user to jump from the user's current state to an idealized state. It may prove more tolerable for the user if the program interpolates. For example, let us imagine that a given user's ultradian peaks might normatively lie 120 minutes apart, but after years of endurance exercise and over-caffeination, they might last 240 or more minutes. In this case, this program may introduce a schedule that progressively decreases the length of ultradian rhythms each day or each week: for example, from 240, to 220, to 200, to 180, and so forth, until the user reaches a more normative 90-120 wavelength. This is a computationally intensive process as it requires substantial knowledge of the user and adjustment of a normative prescribed program. In addition to the standard protocol, therapeutic methods applied to users with conflicting or disrupted or elongated ultradian rhythm might include: periods of continuous monitoring; more precise reporting/monitoring of wake up times; more precise reporting/monitoring of meal times; more precise scheduling of stressor sessions to locate/embed them in the ultradian rhythms that the program attempts to reinforce; and nap training—with emphasis on short naps every day; may advise user to reduce caffeine consumption.

Many mental health professionals struggle to assess when a patient is emotionally activated, when that activation is tolerable versus when it might produce backlash, or when that patient might be receptive to deeper (sometimes unpleasant) counsel. This is understandable, because therapists rely on the patient's self-reporting and “affect” to assess the patient's emotional state. The difference between inner experience—the emotion itself or “feeling”- and the outer expression—the “affect”—can contribute to ruptures in the therapeutic alliance and prompt premature termination of treatment. When a mental health professional misjudges the user's emotional state, the professional might push harder than the patient can tolerate or might continue advancing an incorrect formulation. Many patients feel hesitant to engage in conflict with, or express disappointment in, their therapist. Failure to accurately assess the patient's inner state may contribute to ruptures in the therapeutic alliance and prompt premature termination of treatment. Improving ANS modulation is correlated with reduced risk of many psychological disorders and with improved emotional modulation and improved cognitive flexibility. The improved emotional modulation may allow both the patient and the therapist to move into and out of activated states quickly and may improve tolerance of unpleasant activated states. The improved cognitive flexibility may improve their abilities to mentalize and to simulate other worldviews or other interpretations of an otherwise-activating situation. There is lacking, inter alia, a program that comprehensively attempts to marry this view and re-regulating of the ANS with psychotherapeutic work. A tailored version of the program, with optional additional hardware and software and training specific to this application, may provide value to both mental health professionals and to patients. In addition to standard protocol, therapeutic methods related to this specific application include: training the therapist to use a paced, advance and retreat, progressive overload/diverging oscillatory model rather than a model of constant pressure; training the therapist to use specialized hardware and software to monitor the patient's ANS state in real-time; training both the patient and the therapist to use and interpret morning readings to influence pacing (e.g., days with relatively-lower RMSSD scores might produce gentle reflective conversations about what produced the low scores, whereas days with relatively-higher RMSSD scores might focus on more difficult discussions requiring uncomfortable activation); training the therapist to retreat even when the patient does not require it. A recommendation may be to float an idea or feeling, invite the cognitive flexibility and discomfort required to experiment with the idea or feeling, and then retreat to safety. Training the therapist to offer substantial periods of safety in between these moments is critically important, directly analogous to seeking deep recovery baselines in between “moments” during stressor sessions. Training both the therapist and the patient to recognize the signs—both somatic and ANS—of exhaustion and end a session with safety in order to avoid destructive ruptures. Some ruptures are inevitable and can even be helpful, but this pacing can help make sure the specific stress applied is constructive/hormetic rather than destructive. If the user has exhibited personality disorder issues and/or hyperreactivity, specific emotional modulation sessions may be prescribed as part of the program and/or coordinated with the mental health professional. During these sessions, the algorithm would assist in moving into and out of emotional states to train the modulation flexibility required for other psychotherapeutic work. This is conceptually analogous to training a user to strengthen their biceps, so that they can later lift heavy things more easily. Experiment with session scheduling—early morning if patient is hyperreactive, afternoon before dinner if hyporeactive.

Programs may relate to psychotherapy, psychoanalysis, cognitive behavioral therapy, dialectical behavior therapy. Therapy may include, for example: pacing with daily readings and treatment modification; training the therapist; safety training; hyperreactivity and slope training/emotional modulation sessions; psychotherapy session scheduling—schedule in early morning if hyperreactive, afternoon before dinner if hyporeactive. Training safety is a variation within the program, assisting a user to establish a deep recovery. Training safety may be done in partnership with an emotional specialist within the program or as adjunctive therapy tied to professional psychotherapy. The program may be modified to use concepts of accepted best practice within psychotherapy (i.e., quiet and attuned presence with a trained other) to explore how a state of deep safety might feel and how to return to and tolerate that feeling. Training safety may be used in concert with a version of HRV biofeedback training to confirm the parasympathetic shift.

Nap training is where a pattern of rest is established during the daytime. A user does not need to experience deep sleep or sleep for the entire length of time to benefit from this. This kind of training will slowly introduce and reinforce patterns of rest at healthy times, reinforcing proper ultradian rhythms. Napping includes a top priority nap, which occurs within 30 minutes after breakfast or lunch, is 10-50 minutes long, and happens every day. Even if the user is not tired or is unable to sleep, user should lie down in the dark and quiet, ideally with a contoured eye shade, for the prescribed length of time. It is also acceptable to reserve 10-30 minutes of more-quiet time and activity following the meal.

In some embodiments, the methods, systems, and computer readable mediums relate to generating programs for diagnosing and quantifying how the autonomic nervous system may develop specific abnormal patterns in response to particular illnesses, such as cancer, and for customizing a pattern of stressors that may assist in re-normalizing those patterns. In this case, therapeutic methods might include: reduced apnea sessions, increased periodic coordinated fasting, moving to a lower elevation or more equatorial region.

Aspects relate to systems and methods for diagnosing and quantifying autism spectrum disorder (ASD), oppositional-defiant disorder (ODD), similar symptoms. One interpretation of ASD and ODD is they represent dysregulated inner ANS states. Evidence exists in other studies that shows improving ANS regulation can improve real world functioning for children with ASD. Adherence will prove a key challenge, so the method may adapt the program's built in user profile efficacy testing to account for optimizing adherence on a per-user basis. That is, if the method determines that interventions A, B, and C may all prove effective interventions for a user subgroup, a program may be provided to prove or disprove efficacy of each intervention for an individual user. In this case, the program might also apply testing to see which of these interventions a particular user is most likely to adopt and adhere to, thus maximizing efficacy. This adjustment can be applied both on a user subgroup and an individual user basis. In addition to a standard protocol, therapeutic methods may include: focus on ANS-quieting; focus on somatic experiencing; empirically test elements to maximize adherence; focus on user's specific goals in their plan; and integrate case notes.

Further aspects relate to systems and methods for diagnosing and quantifying how the autonomic nervous system may develop specific abnormal patterns. In an example, hyporeactivity in an initial stress response to particular exercise routines and is indicated by a shallow recovery slope and disorganization in the user's recovery baseline. For promoting an appropriate stress response, an appropriately-rapid recovery slope, and a strong coherent repair period, a protocol for diagnosis may include: evaluation as likely hyporeactive; evaluation as likely disorganized in recovery; excise other chronic stressors; focus on slope more than absolute targets; focus on placing sessions within ultradian rhythms.

Childhood Sexual Abuse (CSA), Post-Traumatic Stress Disorder (PTSD), trauma, bipolar 2, and hypomania aspects relate to systems and methods for diagnosing and quantifying how the autonomic nervous system may develop specific abnormal patterns. For example, hyperreactivity in initial stress responses, level or shallow recovery slopes, and disorganization in recovery baselines may be identified in response to PTSD and childhood abuse. Programs may be generated using the methods and systems herein to promote appropriate stress responses, appropriately-rapid recovery slopes, and strong coherent repair periods. For users with the above conditions or diagnoses or experiences, in addition to a standard protocols, therapy may include: additional hypoxia plus exertion combination moments; distress tolerance training; safety training; or emotional breadth modulation.

Autoimmune and immune related aspects may relate to systems and methods for diagnosing and quantifying how the autonomic nervous system (ANS) may develop specific abnormal patterns (e.g., premature exhaustion, chronic inflammation) in response to autoimmune conditions, and for promoting more appropriate ANS functioning and hopefully symptom improvement and better functioning within the context of the preexisting autoimmune conditions. Therapeutic methods and protocols may include: emotional depth modulation; emotional breadth modulation; additional rest days; or increased temperature modulations.

In some embodiments, systems and methods for diagnosing and quantifying how the autonomic nervous system (ANS) may reflect certain unexpressed emotions or stress responses that lack fluidity. The systems and methods may generate programs for a user to promoting that fluidity, thereby improving ANS functioning and possibly alleviating symptoms. A protocol may include: focus on somatic experience and journaling; increase in emotional modulation session; distress tolerance training; emotional breadth modulation sessions; void training.

In some embodiments, systems and methods may accounts for cognitive decline, dementia, Alzheimer's, or Parkinson's and include modified programs for diagnosing and quantifying how the autonomic nervous system (ANS) may reflect certain rigidity or failures of autophagy, contributing to increased susceptibility for certain neuromuscular and cognitive illnesses. The methods may include program modifications for promoting healthy ANS multi-levered flexibility, thereby improving ANS functioning and possibly alleviating symptoms. In addition to a standard protocol, therapeutic methods may include: PCF—periodic coordinated fasting; metabolic/nutrient flexibility; emotional breadth modulation. In some cases, this may include elements of the program for users under the “GERD/BMI/Weight/Diabetes” section.

Aging may be described as the human body's progressive inability to self-repair or to manage externally damaging effects. This may explain why RMSSD decays exponentially with age, and why RMSSD remains one of the strongest non-invasive correlates with longevity. The diagnostic methods here involve using ASD compared to a peer age and sex cohort, and assessing the user's ANS function versus the function typical for users of that age and of other ages. This provides a relatively non-invasive “biological clock” metric through which to diagnose and measure aging. This can be supplemented and verified with blood tests that confirm known correlates of inflammation and aging. In addition to the standard protocol, therapeutic methods might include: ongoing comparison of the user's chronological age with biological age; ongoing testing at intervals with other metrics that confirm relative age; specific celebration of “unbirthdays” or “reverse birthdays.”

In an initial COVID infection, programs may be modified to support the user's body, brain and medical team to mount a sufficient immune response to repel the infection. Therapeutic methods might include, in addition to the standard protocol, using ASD to provide 1-3 days advanced notice of pending symptom onset, increased temperature modulation sessions, decreased exertion sessions. In the middle COVID (beginning 1-3 weeks post-infection) phase, the program's goal may be to balance a continued fight against the infection with modulating the immune system's hyperreactivity. Therapeutic methods during this phase may include: increased temperature modulation sessions; decreased exertion sessions; some apneic or hypoxic sessions, assuming the user's respiratory system allowed for the sessions. Regarding Long COVID, side effects may include a dysregulation of the user's immune system, affecting the body's ability to fight off future non-Covid illness or re-activation of latent COVID, and/or dysregulate its parasympathetic nervous system, affecting the body and brain's ability to self-repair. One challenge of Long COVID is that many users suffer from lung damage, postural orthostatic tachycardia syndrome (POTS), and general exhaustion. This precludes the intense exercise and/or hypoxic sessions that might typically be applied through a standard program. Another challenge is that Long COVID manifests differently in different patients; this means it may be hard to standardize treatment. In these cases, by re-regulating the ANS, the program may support the body and brain's abilities to self-manage and self-repair. In addition to the standard protocol, therapeutic methods may include: increased temperature modulation sessions; reduce intensity of the standard program; increased rest periods, PSA breathing and focus on recovery depth; use the empirical per-user and per-user-profile-subgroup testing used with respect to adherence for ASD populations and apply them to determine efficacy and tolerance of different interventions.

The program may also include compound stressors in small magnitudes/intensities that, through constructive interference, add up to acute stressors. In other words, the program may simultaneously combine relatively mild/gentle apnea with mild/gentle exertion and mild/gentle temperature modulation. Any one of those stressors might only register 1 or 2 on a 1-to-10 scale, and may be well tolerated by a user with Long COVID who has a limited capacity. By applying those stressors simultaneously, however, the program might be able to apply them in a way that the user can tolerate yet still proves effective in producing the necessary stress response. The algorithm may use the progressive overload patterning of the standard protocol to quantify and time prescribe stress patterns that are tolerable and non-injurious, rebuilding the sympathetic and parasympathetic nervous system range the same way a body builder might rebuild lifting capacity after knee surgery.

A patient's sensitivity to a treatment or drug may permit the prescription of a lower dose—and may therefore reduce the risk of adverse side effects. Improved ANS function can limit side effects and improve tolerance to some drug regimens (e.g., chemotherapy). The diagnostic method, in this case, would be to correlate measures of ANS modulation (e.g., RMSSD) with other measures of drug reactivity/sensitivity; this can be reported in real-time or retrospectively and shared with medical professionals. In addition to the standard program, therapeutic methods may include: integration with self-reporting regarding side effects in the program check-ins and in the wellness plan; integration with ANS reporting to the medical professionals; where possible, ongoing testing to see if ASD may provide proxies for drug efficacy or drug sensitivity in between medical appointments. Additional percentages of stressor sessions assigned as temperature modulation (for immune or autoimmune) or emotional modulation (for pain) or hypoxia/apnea (for hyperreactivity) depending on the specific affect modifications desired depending on drug regime.

A program aspect may include session mapping via inputs, personal history, diagnostic tests, traumas, timing, levers, specific actions, muscle groups, HR reactivity, exhaustion capacity, available equipment, minor and major rhythm reestablishment and reinforcement, ultradian reinforcement, lunar forecast, morning forecast, and real time in-session feedback. The process of “session mapping” involves taking inputs from an individual's daily readings, personal history (including traumas), diagnostic tests run on the individual's ASD, the user's observed ASD and sorting into user profile subgroups (including reactivity, RSA, etc.), minor and major rhythm analysis (e.g., does the user have elongated or conflicting ultradian rhythms or report difficulty sleeping?) and building out a map for what an individual session might optimally require. The process may attempt to time the session optimally given the lunar cycle, the user profile (some users may experience better reactivity, or too much reactivity, before lunch, versus before dinner), and within the context of that individual's ultradian rhythms. The system may try to assess how many moments to include in the session, what kind of variability or “noise” to introduce, and which stressors to apply in each moment. If exertion is one of the stressor types, the process attempts to assess what capacity for work (including maximum stress targets and muscle/weight and mobility targets), what mobility or injury restrictions are noted in the user profile, and which muscle groups have been exhausted in recent past sessions, and what equipment the user has available. If adding another stressor, the process may also adjust downward the exertion intensity so that the combined stress intensity (see constructive interference) does not prove injurious.

In addition to the practical application (i.e., the diagnostic and therapeutic methods unto themselves) of the above algorithms, there are combined underlying understanding of and ways to assess the ANS and report on its relative characteristics. Current ANS metrics typically fall into two categories: (a) Normal-curve-fit models for RMSSD or InRMSSD scores, some of which are then scored 1-100, and some of which are also scored versus population averages; and (b) Power spectrum scores, consisting of metrics separating RMSSD and HRV statistics into components like High Frequency, Low Frequency (“HF/LF”) ratios, Total Power, etc. Additional metrics are provided that assess a user's ANS in detail, including specific reactivity, RSA, and waveform signal identification and extraction characteristics. Individual user profiles and user subgroup profiles from these metrics may be built. Applications might include but are not limited to using these profiles to help assess somebody's health, forecasting long-term care need, their drug dosage sensitivity, emotional reactivity, and other specific aspects of risk (cardiovascular vs mobility vs pulmonary, for example). Some metrics and signals that might contribute towards these metrics include, but are not limited to: upward momentum; exhausted moment; peak exhaustion; increasing baseline; head and shoulders analysis; fractality; fractal coherence—major/minor conflicts; conflicting rhythms; and oscillatory start/stop signals. Oscillatory mechanics/dynamics include any specific interventions and a new calculus used to affect the system, recognizing that the ANS is not steady state. An upside breakout is an oscillation more than three beats per minute (bpm) or at least 5% higher than standard local maximum oscillation. It is common for a seated user's HR to enter an oscillatory range, often plus or minus just a few beats per minute (BPM). An upside breakout signal occurs when HR moves higher than the typical oscillatory range preceding the signal. A diverging oscillation is a sequence of rolling oscillations, wherein each new peak is higher than the last peak, and wherein often the delta between local prior minimum and local new peak high continues to grow.

A peak escalation is the rate of increase, or curve fitted, from peak of one acute moment to the next over the course of a session. Circadian is a daily rhythm. Lunar or infradian is a monthly rhythm. Ultradian is an intraday rhythm, typically 90-120 minutes in length. Underlying (or major) rhythm is a longer wavelength larger scale rhythm that influences and is simultaneously influenced by shorter-wavelength rhythms. An example of this might be how ultradian rhythms can affect and be affected by the underlying circadian rhythm. This fractality applies up the scale, all the way to the organism's whole life cycle.

Overlying (or minor) rhythms are a shorter wavelength rhythm that influences and is simultaneously influenced by longer-wavelength rhythms. An example of this might be how circadian rhythms can affect and be affected by overlying ultradian rhythms. This fractality applies down the scale, all the way to at least the organism's ECG rhythms.

Embedding or locating may be used in a program to apply stressors or recovery periods at specific times of day, week, month, season, or year, in order to enhance the fractality or self-similarity of rhythms at other scales. In one modified program example, a user may be advised to work out before breakfast one week, and before lunch another, in order to reinforce ultradian rhythms. In another modified program example, a user may be recommended to fast near the new moon to reinforce lunar cycles. Fasting may be recommended for 24-36 hours during a specific time window in order to reinforce lunar rhythms. The highest risk period for Seasonal Affective Disorder and strokes is late-winter/early Spring; during this time, a program may suggest clients in northern climes use more temperature oscillations. Temperature modulation may include divergent contrasts in body temperature, such as by a cold shower, which may be used as minor oscillatory reinforcer. Temperature modulation can reference the application of heat during moments, or specific during the beginning of moments, to increase acute stress response.

A Pull modified program may be recommended the time from last-quarter-moon to the day before new moon, during which the following may be included in the program: more sessions; up to 9 acute moments per session, with very short—4-20 second long each—acute moments of exertion of lighter intensity, without apnea. The program may include meal plans. In one example, the biggest meal may be breakfast. The program may advise the user to sometimes skip breakfast on days without exertion sessions, often the phase the user might describe as depressive.

A Drive/Build modified program may be recommended the time from new moon to the day before first-quarter-moon, during which the following may be prescribed: reduction in a number of sessions from the Pull phase, with exertion moments of increasing duration and intensity, starting at 10 seconds and ending at 60 seconds, with possible apnea, added either to the beginning, end, or all of the session's moments. Depending on the user's personalized profile, the biggest meal will typically be lunch and the user will sometimes skip dinner on days without exertion sessions, often the phase the user might describe as feeling best balanced.

A Push modified program may be recommended the time from first-quarter-moon to the day before full moon and may include: reduction in a number of sessions from Pull and Build/Drive phases, just 3-5 moments of acute exertion per session, all moments hard and 45-60 seconds long, with possible apnea, added either to the beginning, end, or all of the session's moments, depending on the user's personalized profile. The program may include a recommendation for the user's biggest meal to be dinner, often a phase described as most chaotic, stressed, stretching for the user.

A Drift/Glide/Grow modified program may be recommended the time from full-moon to the day before last-quarter-moon, and may include: no acute exertion moment session, the user does not exercise too vigorously, the user is encouraged to eat three meals a day intuitively, until they feel 80% sated. The modified program may include insight that this phase is often the phase users describe as dizzying, anxious.

A program application may use an ID profile/dongle, which is either a software or hardware identifier of a user and communicates the user's specific patterns and session needs to showers, workout equipment, wellness advisor, smart equipment, other devices, or some combination thereof.

### ANS Regulation System Architecture

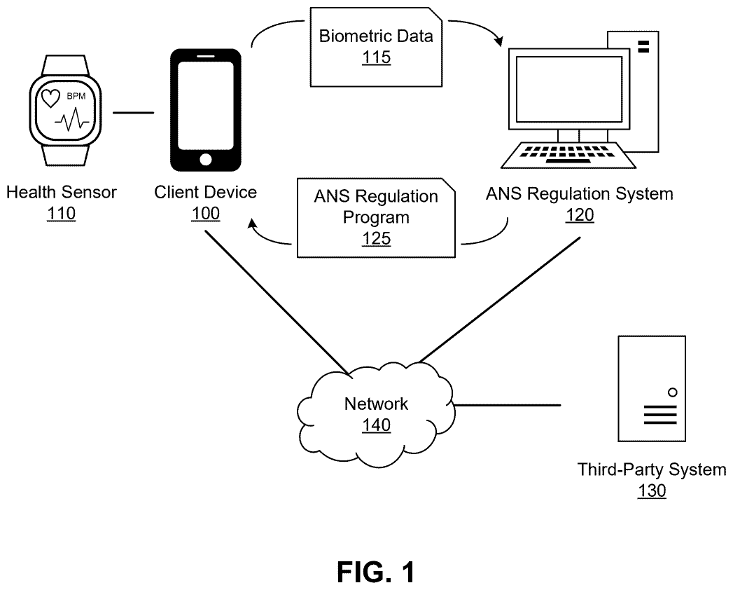

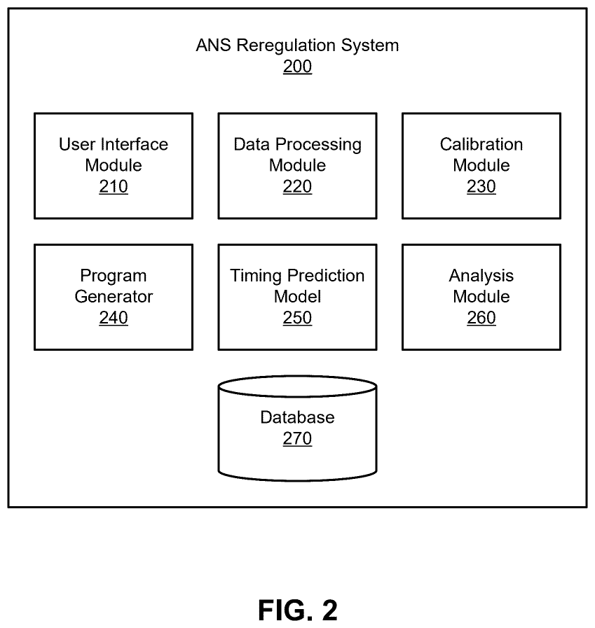

FIG. 2 is a block diagram illustrating an architecture of an ANS regulation system 200, according to one or more embodiments. The ANS regulation system 200 is an embodiment of the ANS regulation system 120. The ANS regulation system 200 maintains one or more ANS regulation programs to be used by an individual, e.g., via their client device (e.g., the client device 100). In some embodiments, the ANS regulation system 200 maintains real-time communication with the user's client device, providing data in real-time (or near real-time) to the client device for performance of an ANS regulation program. In other embodiments, the ANS regulation system 200 may generate parameters for performance of the ANS regulation program, e.g., based on the analyses of the user's health data. The ANS regulation system 200 provides the parameters to the user's client device for performance of the ANS regulation program.

In one or more embodiments, the ANS regulation system 200 includes a user interface module 210, a data processing module 220, a calibration module 230, a program generator 240, a timing prediction model 250, an analysis module 260, and a database 270. In other embodiments, the ANS regulation system 200 may include additional, fewer, or different components than those listed in FIG. 2. In other embodiments, the functionality of each module may be disparately distributed across the components than as described herein. In other embodiments, some or all of the functionality of each module may be performed by the user's client device.

The user interface module 210 generates a user interface for presentation by the user's client device. The user interface module generates a user interface leveraging real-time and historical health data, including data streams from one or more health sensors. The user interface module 210 may generate the user interface to display relevant information regarding the user's current health state, e.g., based on the health data, results of analyses by the ANS regulation system 200, results of one or more previously performed ANS regulation programs, or some combination thereof. The presented information may include visualizations such as charts, graphs, and personalized dashboards that provide insights into key health metrics like heart rate, sleep patterns, activity levels, and biometrics. The user interface may, in other embodiments, present audio content, haptic content, text, augmented reality content, virtual reality content, mixed reality content, or some combination thereof. The interface also facilitates user interaction through various input mechanisms, such as interactive questionnaires, surveys, and customizable goal-setting tools. Users can provide feedback, adjust preferences, and actively engage with the application to personalize their health journey. The user interface module 210 may track the input by the user, e.g., in the database 270.

The user interface module 210 may also generate the user interface to present an ANS regulation program being performed. In such embodiments, the user interface module 210 may generate elements for inclusion on the user interface to guide the user through the ANS regulation program. For example, the user interface module 210 may generate visual elements for cueing the user on timing of performing one or more actions as part of the ANS regulation program. In another example, the user interface module 210 may generate audio elements presented in conjunction with the visual user interface for cuing the user on the timing. In one or more embodiments, the user interface module 210 may leverage a vocal synthesizer to generate vocal tracks for presentation with the ANS regulation program.

The data processing module 220 processes data received by one or more health sensors and/or one or more third-party systems (e.g., the third-party system 130). Biometric data streams may be high-volume data, inherently noisy and variable, and sensitive in terms of user privacy and security. The data processing module 220 may perform processing techniques to address these characteristics. For example, the data processing module 220 may leverage signal processing techniques to identify informative features in the data streams, while filtering out noise or artifacts arising from the data capture methodology. The data processing module 220 may further leverage machine learning techniques (e.g., classification, regression, anomaly detection) to perform the various processing techniques. In one or more example implementations, the data processing module 220 may perform ECG processing which may involve some combination of filtering noise, extracting features from R-R intervals, and classifying heart rhythms from the ECG signal.

In one or more embodiments, the data processing module 220 may synchronize formatting across disparate data streams. For example, the ANS regulation system 200 may receive one data stream formatted in one manner by the health sensor used to capture the data stream and may further receive another data stream formatted in a different manner by a different health sensor used to capture the data stream. To synchronize the different formats, the data processing module 220 may transform the data from one data stream into the format of the data in the other data stream. In other embodiments, the data processing module 220 may transform each data stream into a common format.

The data processing module 220 may, in one or more embodiments, identify features in a shared feature space from both data streams, thereby empowering comparability of the two data streams. For example, the data processing module 220 may extract a first heart rate variability (HRV) timeseries from an ECG signal captured by an ECG sensor and a second HRV timeseries from a heart rate signal captured by a heart rate sensor. To extract the HRV timeseries from the ECG signal, the data processing module 220 may perform R-peak detection by identifying local maxima in the ECG signal, representing the strongest electrical activity in each heartbeat. The data processing module 220 may perform a R-R interval calculation to identify a temporal difference between each successive R-peak. The data processing module 220 may quantify the variability of the temporal difference between successive R-peak's in the time domain, e.g., leveraging root mean square successive differences (RMSSD), standard deviation of all normal-to-normal intervals (SDNN), another frequency-domain analysis, or some hybrid thereof. In a similar manner, the data processing module 220 may calculate the HRV timeseries from the heart rate signal by identifying inter-beat intervals. From the inter-beat intervals (i.e., the temporal difference between success beats), the data processing module 220 may calculate the variability in the heart rate over time, i.e., the HRV timeseries. In one or more implementations, the HRV timeseries derived from a heart rate signal is coarser than the HRV timeseries derived from an ECG signal. However, the ECG signal is more challenging to obtain, typically using several electrodes coupled to different positions on the user.

The calibration module 230 generates a calibration profile for a user based on the user's data. The calibration module 230 may build the calibration profile based on the processed data, e.g., by the data processing module 220. In some embodiments, the data includes biometric data on a user's response to a historical ANS regulation program. Such data may indicate the user's autonomic nervous system's response to one or more stressors in the ANS regulation program. The calibration module 230 leverages such information to build the calibration profile representing a current state of the user's ANS. The calibration profile may characterize various aspects of the user's ANS, e.g., which may include HRV, blood pressure, sweat production, temperature regulation, respiratory function, pupil dilation, activity of the sympathetic nervous system responsible for the fight-or-flight response, the parasympathetic nervous system responsible for the rest and digest functions, etc. In one example, the calibration profile may specify the user's statistics on the user's HRV compared to percentile ranges for a general population of users. As a numerical example, the calibration profile may indicate that one 20-year-old user has an HRV value of 80, which is around the 50th percentile for 20-year-olds. The calibration profile may further characterize the user's ANS response to various stressors, e.g., physical exertion, respiration, temperature, light, sleep, nutrition, emotion, etc. For example, in response to a respiratory control exercise, the calibration module 230 may track one user's heart rate response to various sessions to build an average response curve for the user. That user's response curve can be distinct from other users. The calibration module 230 may store the calibration profile in the database 270.

The program generator 240 generates an ANS regulation program for provision by a client device. The ANS regulation program includes one or more actions for regulation of the user's ANS. In one or more embodiments, the ANS regulation program may be performed over a single session, e.g., within a 30-minute period, or over a plurality of sessions, e.g., a 15-minute period each morning. The program generator 240 may generate the ANS regulation program tailored to the individual's health data. For example, the program generator 240 may target, as one of its objectives, increasing the user's HRV. The program generator 240 may leverage a template for generating the ANS regulation program. The template may include a list of one or more stressors that may be included, an ordering of the one or more stressors, duration of the one or more stressors, etc. The program generator 240 builds the tailored ANS regulation program by modifying the template to suit the user's current physiological state. For example, the user's HRV may be particularly low (e.g., in the lowest decile among users of similar demographic background). As such, the program generator 240 may start with a relatively lower stress program. This may entail leveraging less actions in the program, lessening an intensity of the actions in the program, compacting the program, etc. The program generator 240 may adjust one or more different characteristics in the ANS regulation program to tailor the program to the user. In another example of tailoring the program, the program generator 240 may bias towards selection of one type of stressor that is most effective at regulation of the user's ANS. Quantification of regulation of the user's ANS may be performed by the analysis module 260, e.g., movement in the user's HRV. The program generator 240 may further include, in the ANS regulation program, parameters for performance of each exercise. For example, the parameters may include instructions for timing the

In one or more embodiments, the program generator 240 generates the ANS regulation program to operate as a state model. The state model includes the one or more actions to be performed by the ANS regulation program as states in the state model. To transition between the actions, the state model leverages one or more transition criteria. Upon satisfaction of the transition criteria, the state model progresses to the next state, i.e., the next exercise. One example transition criteria seeks to hit a target HRV for one stressor exercise. As such, to transition to the next exercise, the state model would evaluate whether the user's HRV meets the target HRV. If achieved, the state model would transition to the next state, i.e., the next exercise. In one or more embodiments, the state model may include additional logic operating on the transition criteria. For example, the logic may specify that two or more of the transition criteria need to be satisfied prior to progressing to the next state. In another example, the logic may specify that if at least one transition criteria is satisfied, the state model would progress to the next state. Example transition criteria may include: achieving a target metric in the biometric data, iterating a maximum number of iterations for a state, receiving input from the user to transition to the next state, etc. In some embodiments, the state model may transition to different states based on the transition criteria satisfied. For example, if a target metric is achieved, the state model may transition to a second state with one type of stressor at a high intensity level. If, however, the maximum number of iterations are exhausted, the state model may transition to a third state with the one type of stressor at a low intensity level, lower than the high intensity level.

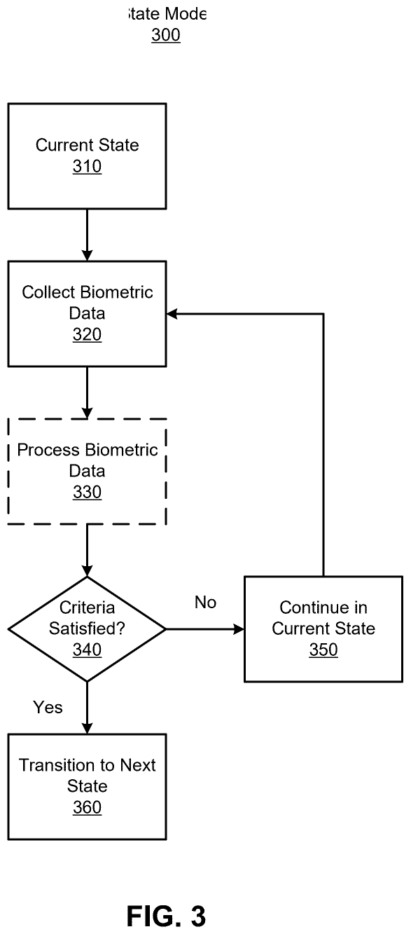

FIG. 3 illustrates an example process for a unit in a state model 300, according to one or more embodiments. The state model 300 includes one or more states and transitions between the states, which together define the ANS regulation program performed by the client device in regulating a user's ANS. Each state in the state model may include at least one activity as part of regulating the user's ANS. For example, one state may stress the user's ANS, another state may recover the user's ANS, yet another state may evaluate the user's ANS. The state model may include one or more repetitions of the example unit shown, to transition through the various states in the state model 300, i.e., until an end state is reached, which concludes the ANS regulation program. A client device of the user transitions performs the ANS regulation program to regulate the user's ANS, leveraging the state model 300 to transition between different states to complete the ANS regulation program.

In the example unit, the state model 300 starts at the current state 310, e.g., which may be an exercise to stress the user's ANS. The client device collects 320 biometric data as the user is in the current state 310. The client device may process 330 the biometric data (e.g., via techniques described under the data processing module 220). The client device evaluates whether criteria are satisfied 340 to transition to the next state. For example, if the user's biometric data indicates the user's ANS has achieved a target metric to transition to the next state, then the client device may identify the criteria as satisfied. If not, the client device continues 350 in the current state. This may entail repeating an exercise for the user. If the criteria have been satisfied, the client device transitions 360 to the next state. The client device progresses through the state model 300 until an end state is reached, e.g., all other states (including their actions) have been completed. Upon conclusion of the state model 300, the client device may indicate the completion of the ANS regulation program. In other embodiments (not illustrated), the state model 300 may include branched pathways. For example, one state may branch into two or more different states, depending on the outcome of the upstream state. In other embodiments, the state model 300 may include parallel processing, where the ANS regulation program may simultaneously leverage two or more states. For example, a breathing timing state (inhalation, hold, or exhalation) may be concurrent with an exertion state (workout, recover, etc.).

Referring back to FIG. 2, the program generator 240 provides the ANS regulation program to the client device for performance with the user. In some embodiments, the program generator 240 provides the program to the user interface module 210 to be presented in the user interface. The ANS regulation program may include parameters controlling precise performance of each of the various actions included in the ANS regulation program. In some embodiments, the ANS regulation program may include parameters for guiding performance. For example, one parameter may specify a baseline value for a biometric signal. When the user achieves that baseline value, then the ANS regulation program may begin some action.

In one or more embodiments, the program generator 240 may build out the ANS regulation program on-the-fly, adapting to real-time biometric signals captured by one or more health sensors coupled to the client device. In such embodiments, the ANS regulation system 200 may receive the real-time biometric signals for analysis. Based on results of the analyses, the program generator 240 may adapt the program, i.e., on-the-fly, to better suit the user's current physiological state. For example, perhaps the user is fatigued one day. Such fatigue could cause the user's ANS to perform outside a normal expected range for the user. As such, the program generator 240 may tailor the program to address the user's outlier physiological state.

The timing prediction model 250 determines start times for stressors in an ANS regulation program. In one or more embodiments, the timing prediction model 250 obtains real-time biometric signals from the one or more health sensors to identify start times for the stressors. The timing prediction model 250 may take into account a user's reaction time, i.e., how long it takes the user to begin an exercise following prompting. In other embodiments, the ANS regulation system 200 may provide the timing prediction model 250 to the client device, in conjunction with the ANS regulation program. In such embodiments, the client device may leverage the stressing timing prediction model 250 to identify the start times for stressors in the ANS regulation program. In one or more embodiments, the timing prediction model 250 analyzes biometric signals of the user to determine a pattern in the user's ANS. Based on the pattern, the timing prediction model 250 may identify the start times for a stressor of the ANS regulation program. For example, with a heart rate signal, the timing prediction model 250 may determine a frequency in the oscillation of the heart rate signal. Based on the frequency, the timing prediction model 250 may determine the start time for a stressor to align with an incline in the heart rate signal.

The timing prediction model 250 may also determine end times for stressors, to begin recovery. In one or more embodiments, the timing prediction model 250 obtains real-time biometric signals from the one or more health sensors to identify end times for the stressors. Heuristics may be also applied to identify the end times. In one or more embodiments, the timing prediction model 250 determines an end time for a stressor in response to detecting the biometric data achieving a target value, e.g., a target heart rate. In one or more embodiments, one heuristic specifies a maximum timing for applying a type of stressor (e.g., which may be based on the user's calibration profile). In one or more embodiments, one heuristic specifies a manner in identifying an inflection point in the ANS response, represented by the biometric data, to time the end for the stressor. The timing prediction model 250 may also take into account the user's reaction time in identifying the end times for the stressors.

The analysis module 260 performs one or more analyses on the biometric data obtained from one or more health sensors. The biometric data may be processed by the data processing module 220. The biometric data may be obtained during an observation session, i.e., a session where no stressors are applied as the biometric data is collected. The biometric data may, alternatively, be obtained during performance of an ANS regulation program, i.e., as the user's ANS is stressed and recovered by prompting one or more actions. The analysis module 260 performs analyses to assess the user's ANS. For example, the analysis module 260 may calculate progression of a user's ANS over a period of time. In another example, the analysis module 260 may determine an efficacy score for different characteristics of the ANS regulation program. This may include an efficacy score per type of stressor used. The efficacy score provides insights into the user's ANS, which may be used by the calibration module 230 to update the user's calibration profile. For example, the different types of stressors may be ranked based on their efficacy scores. The analysis module 260 may also identify particular combinations and/or orderings of actions that optimize regulation of the user's ANS. For example, one user's ANS may be more stressed by the provision of a respiratory-based stress exercise following a light-based stress exercise. The analysis module 260 may store the various results of the analyses in the database 270. The analysis module 260 may also provide the results of the analyses to other components of the ANS regulation system 200 for refinement of models, programs, parameters, profiles, etc.

The database 270 stores data used by the ANS regulation system 200. The database 270 may include health data collected for users of the ANS regulation system 200. The health data may include user responses to a health questionnaire, biometric data captured by one or more health sensors, clinical data obtained from a third-party system (e.g., an electronic health records database), or some combination thereof. The database 270 may further store calibration profiles for users, logs of completed ANS regulation programs, parameters for ANS regulation programs, studies implicating ANS regulation with various health application (e.g., from third-party systems), or any other data used by the ANS regulation system 200.

### Example Timing for ANS Regulation Program Stressors

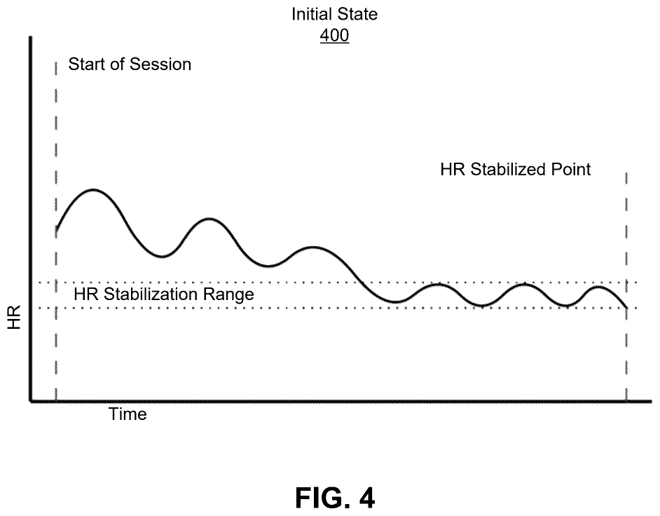

FIGS. 4-13 illustrate example timings for an ANS regulation program's stressors, according to one or more embodiments. In one or more embodiments, a timing prediction model (e.g., the timing prediction model 250) may be leveraged to identify the optimal start times illustrated and described in the various figures. In one or more embodiments, the client device obtains biometric signals from one or more health sensors, with which the client device may identify timings for progressing through an ANS regulation program.

FIG. 4 illustrates identifying an HR stabilized point for an initial state 400 of an ANS regulation program, according to one or more embodiments. The HR shown is collected by a health sensor coupled to the client device. At the start of the session, the user's HR may be more volatile, e.g., due to the user moving around or not having yet settled. As the user's HR begins to stabilize, the client device identifies whether the user's HR is within an HR stabilization range for a minimum duration (e.g., 3 seconds). The HR stabilization range may be based on the user's calibration profile. In some embodiments, the initial state 400 may include additional time limits, e.g., minimum timing of 6 seconds and/or maximum timing of 120 seconds. In some embodiments, identifying the stabilized point includes calculating a slope of the HR over a window of time. If the slope of the HR over a window of time is within some tolerance of zero, then the stabilized point may be identified at the current timestamp. In some embodiments, the rolling HR spread is calculated (e.g., as a difference between the minimum and the maximum values in a window of time). If the rolling HR spread is above a threshold spread, then the user is deemed not stabilized. If the rolling HR spread is within the threshold spread, then the user is deemed stabilized. In some embodiments, the rolling HR spread is evaluated against a population, e.g., if the rolling HR spread falls within a 90^{th} percentile of the rolling spread observed at true triggers in training data. In one or more embodiments, identification of the HR stabilized point is a triggering criteria for progressing to the next state, following the initial state 400.

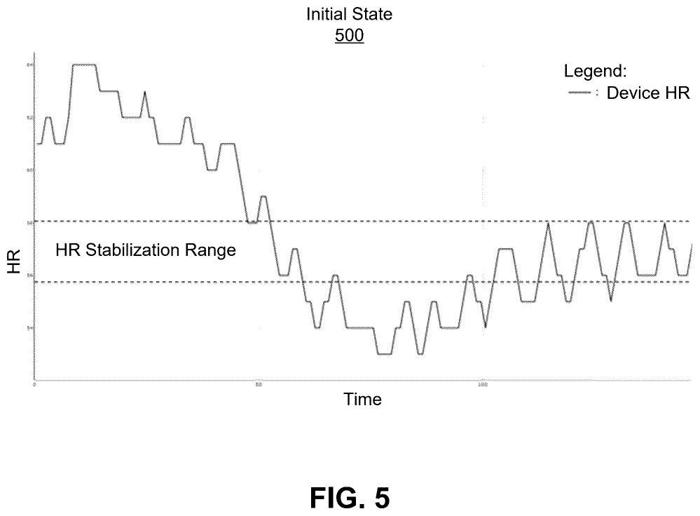

FIG. 5 illustrates identifying the HR stabilized point for example data of a user, during an initial state 500 of an ANS regulation program, according to one or more example implementations. As shown in FIG. 5, the HR is above the HR stabilization range at the start of the sensing, e.g., perhaps the user had not yet settled. The HR then dips below the range, before settling into the HR stabilization range. The stabilized point can be identified, e.g., based on the user's HR within the HR stabilization range for a minimum duration, based on the HR slope within some tolerance of 0, the HR rolling spread within some threshold spread, or some combination thereof.

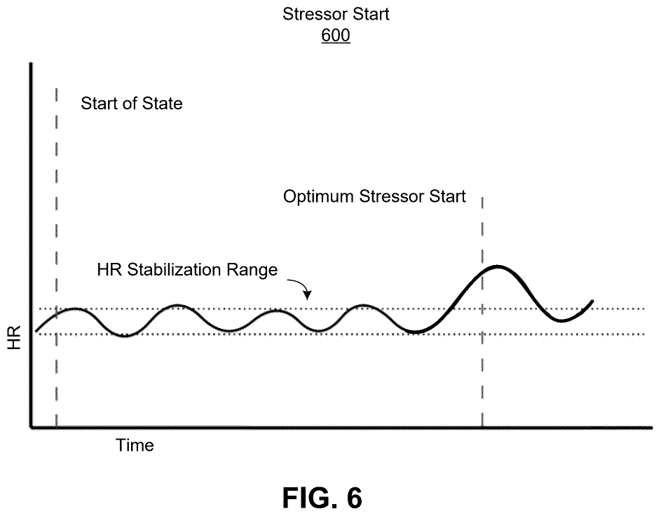

FIG. 6 illustrates identifying a stressor start time 600 during an ANS regulation program, according to one or more embodiments. At the start of the state (assuming following the initial state), the user's HR is stabilized. The optimal start time of the stressor is during an upswing in the user's HR. The user's HR may be sinusoidal. The optimal start time may be determined based on a frequency of the user's HR timeseries and/or based on the user's reaction time. The optimal start time may also rely on one or more other checks. One check may provide temporal limits to starting a stressor, e.g., minimum of 5 seconds and/or maximum of 360 seconds. Another check identifies whether the user is still within the HR stabilization range. The device checks whether the current HR is within some standard deviations of the past HR window of time. The ANS regulation program may be configured to detect an upswing in the user's HR that exceeds the stabilization range, i.e., an upside breakout signal. The ANS regulation program may leverage this upswing to trigger a stressor start.

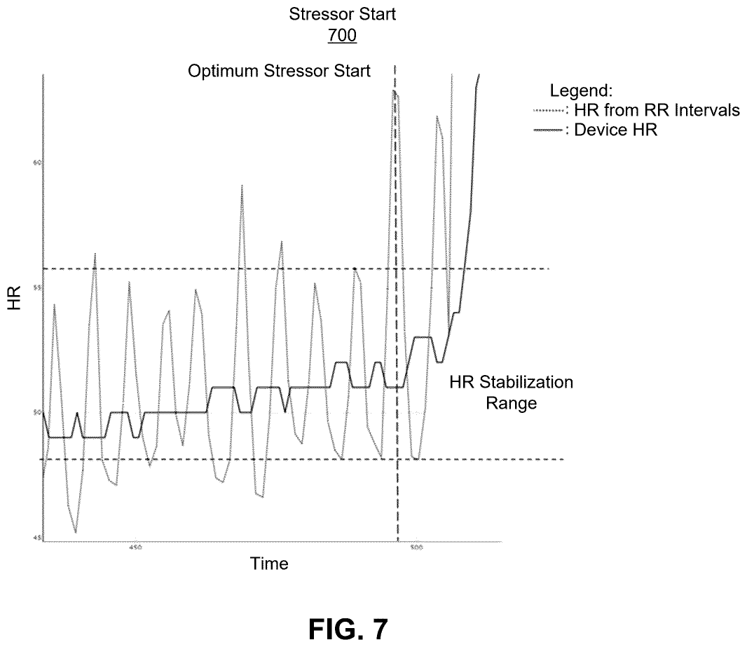

FIG. 7 illustrates identifying a stressor start time 700 during an ANS regulation program, according to one or more example implementations. As shown in FIG. 7, HR may be obtained from two different sources: (1) HR from the R-R intervals, and (2) HR from a health rate monitor. The device performs the checks and identifies the stressor start time 700, i.e., during an upswing in the user's HR. Here there is some discordance between the two HR signals. The device may prioritize one over the other. In response to the stressor start, the user's HR begins to climb, clearly exiting the HR stabilization range.

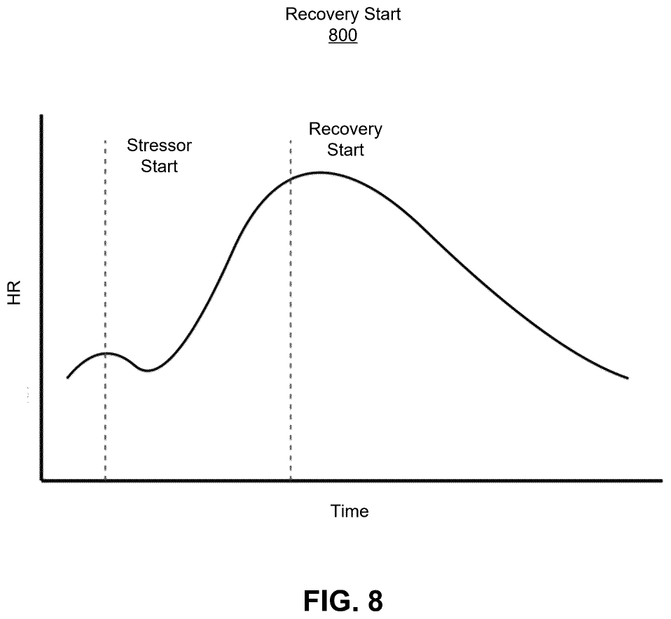

FIG. 8 illustrates identifying a recovery start time 800 during an ANS regulation program, according to one or more embodiments. The stressor has started, resulting in a rise in the user's HR, i.e., as an ANS's response to the stressor. The recovery start time (i.e., end time of a stressor) may be near a target HR. The target HR may be based on the user's calibration profile, e.g., 1.5×, 2.0×, 2.5×, etc. of the median HR of the user's HR stabilization range. In other embodiments, the recovery start time may be based on temporal limits imposed on the stressor. The recovery start time may also rely on one or more other checks. One check may provide temporal limits to starting a stressor, e.g., minimum of 5 seconds and/or maximum of 360 seconds.

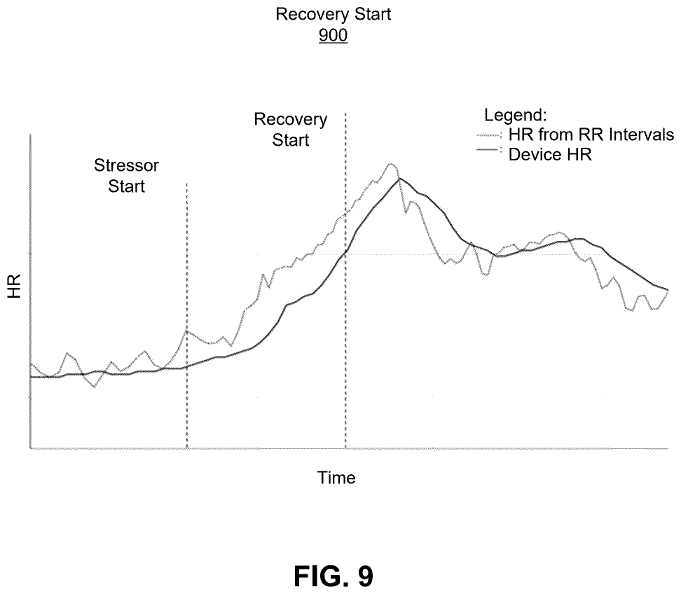

FIG. 9 illustrates identifying a recovery start time 900 during an ANS regulation program, according to one or more example implementations. As shown in FIG. 9, HR may be obtained from two different sources: (1) HR from the R-R intervals, and (2) HR from a health rate monitor. The device performs the checks and identifies the recovery start time 900, e.g., once a target HR is achieved. Here the device may aggregate the HR signals from the two sources. In response to the recovery start, the user's HR may continue climbing, as a residual effect to the stressor, before peaking and then declining. In between stressors, the ANS regulation program may repeat stabilization of the user's ANS.

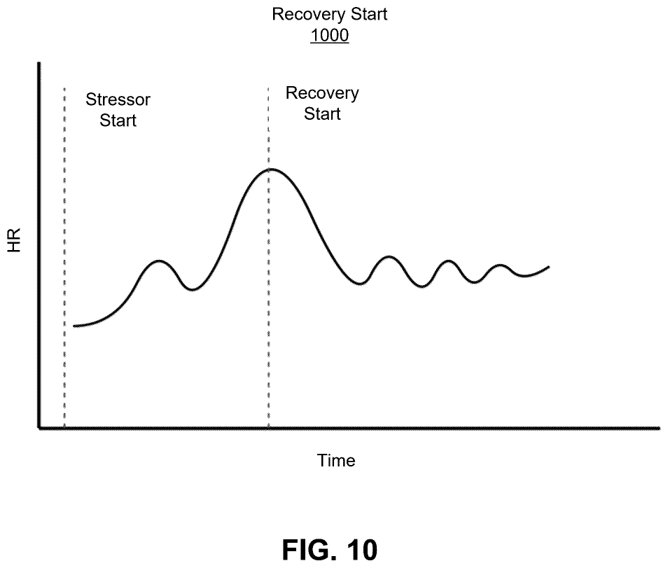

FIG. 10 illustrates identifying a recovery start time 1000 for a respiratory-based stressor during an ANS regulation program, according to one or more embodiments. The stressor may be a prompt for the user to hold their breath. As the stressor starts, the user's HR rises, i.e., as an ANS's response to the stressor. There may be, in general, a brief spike and return to a lower HR as a result of a deep inhalation (as prompted), which is bypassed. At the beginning of the inhalation and hold, the HR slope is steep. As the inhalation prolongs, the HR slope levels and may even begin to decrease slightly. The zero slope (or within some tolerance of the zero slope) is the recovery start time (e.g., exhale time). In some embodiments, the recovery start time may be triggered at a target HR. The recovery start time may also rely on one or more other checks. One check may provide temporal limits to starting a stressor, e.g., minimum of 10 seconds and/or maximum of 30 seconds. These limits may be adjusted based on the user's calibration profile.

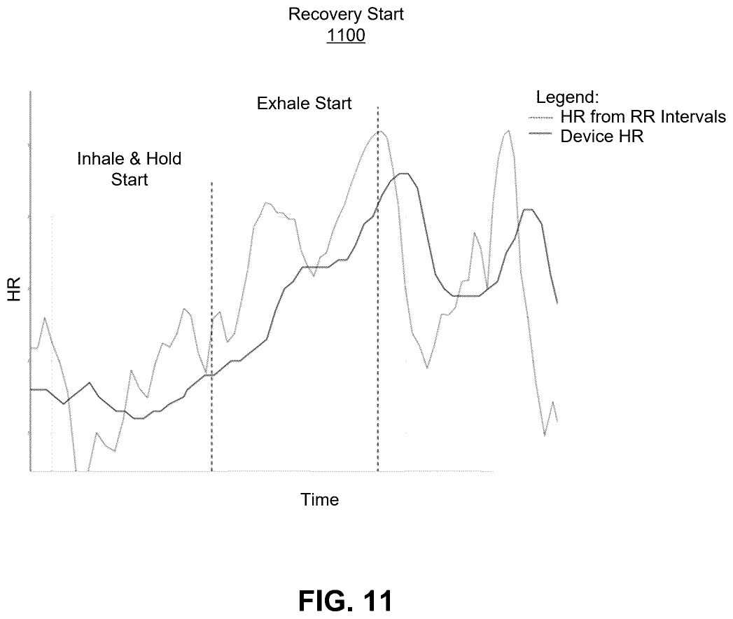

FIG. 11 illustrates identifying a recovery start time 1100 for a respiratory-based stressor during an ANS regulation program, according to one or more example implementations. As shown in FIG. 11, HR may be obtained from two different sources: (1) HR from the R-R intervals, and (2) HR from a health rate monitor. The device performs the checks and identifies the recovery start time 1100, i.e., at the zero slope of the HR and/or when a target HR is achieved. Here the device may aggregate the HR signals from the two sources. In response to the recovery start, the user's HR may continue climbing, as a residual effect to the stressor, before peaking and then declining. In between stressors, the ANS regulation program may repeat stabilization of the user's ANS.

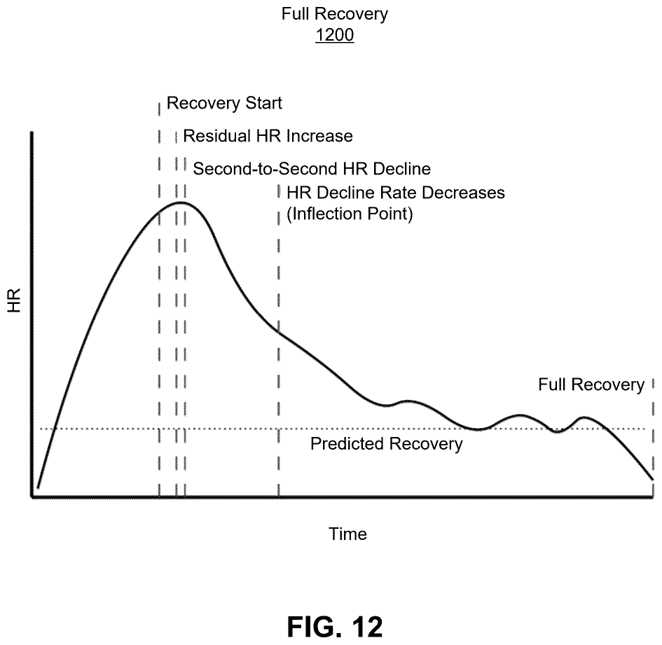

FIG. 12 illustrates identifying a full recovery 1200 following a stressor during an ANS regulation program, according to one or more embodiments. After the stressor has stopped, the user's HR continues to decline back towards a predicted recovery threshold in the HR. At the recovery start, the user's HR may continue to climb, as a residual effect of the stressor. The user's HR peaks then begins to decline. The user's HR declines at a steep rate, initially, before the decline rate tapering (i.e., at the inflection point). The HR continues to decline until the predicted recovery threshold. The predicted recovery threshold may be based on the user's calibration profile, HR values of past stabilization points, etc. The full recovery may also rely on one or more other checks. One check may provide temporal limits to starting a stressor, e.g., minimum of 10 seconds and/or maximum of 480 seconds. These limits may be adjusted based on the user's calibration profile. The rolling slope (i.e., the slope fitted over a window of time) can also be checked against zero. The rolling HR spread can also be checked. If the user's rolling HR spread (i.e., the difference between minimum and maximum values over a window of time) is beyond a threshold spread, then the device may determine the user has not yet reached full recovery 1200. The device may also check the rolling HR spread against population percentiles.

Following a stressor, the device may also check for exhaustion of the user. Decoupling is a phenomenon when the HR does not respond in the same manner as earlier (e.g., the HR does not reach the same maximum (or even near the same maximum) with the same or more effort). In one or more embodiments, the device checks whether the HR peaks of consecutive stressors (of the same type, same intensity, same duration) are indicative of decoupling. If so, the device may detect exhaustion of the user, and trigger an end state of the ANS regulation program.

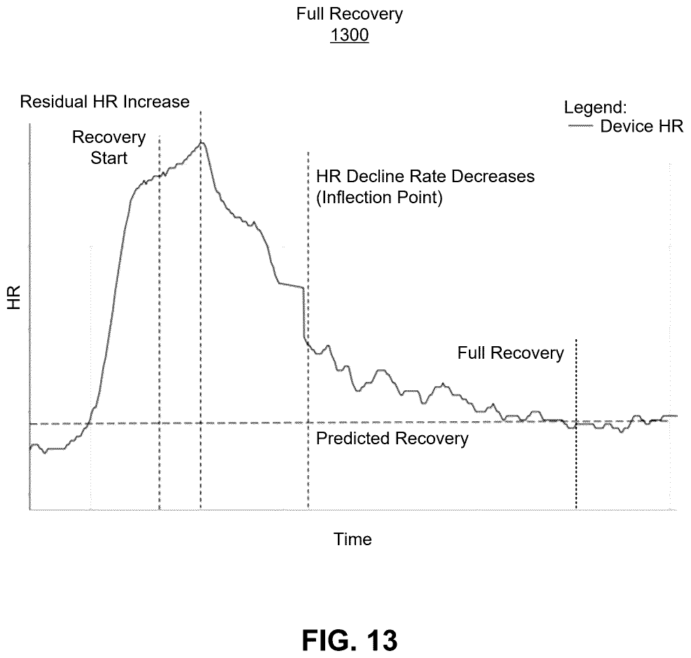

FIG. 13 illustrates identifying a full recovery 1300 following a stressor during an ANS regulation program, according to one or more example implementations. The device performs the checks and identifies the full recovery 1300, i.e., the user's HR reaches the predicted recovery threshold. The device leverages this full recovery identification in between stressors to optimize the stressors in the ANS regulation program. Without full recovery, application of a subsequent stressor may quicken exhaustion, thereby cutting the ANS regulation program short. As such, detection of full recovery is key to prolonging the ANS regulation program to achieve maximal effect.

### Example Methods

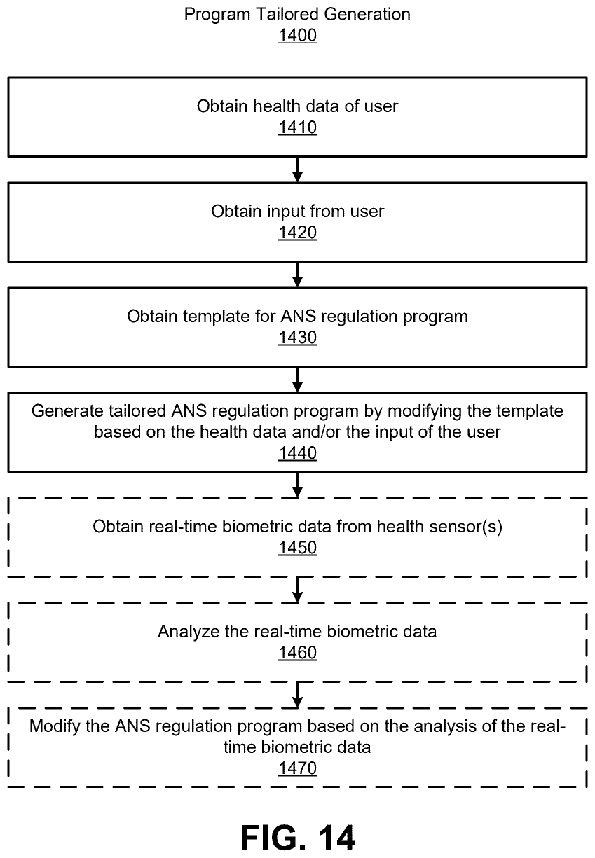

FIG. 14 illustrates a process of generating 1400 a tailored ANS regulation program, according to one or more embodiments. The process of generating 1400 the tailored ANS regulation program may be performed by an ANS regulation system (e.g., the ANS regulation system 120 or the ANS regulation system 200). In other embodiments, the process of generating 1400 the tailored ANS regulation program may be performed in conjunction with input provided by a client device (e.g., the client device 100). In other embodiments, the process may include additional, fewer, or different steps than those listed in the illustrative flowchart.

The system obtains 1410 health data of the user. The health data may include biometric data from one or more health sensors coupled to a client device of the user, e.g., a heart rate signal captured by a heart rate monitor, or derived from R-R intervals from an ECG signal captured by an ECG monitor. Health data may also include logs of past programs completed by the user. Health data may also include clinical data from a healthcare provider, e.g., securely accessed, with permission from the user, from a third-party system. Health data may also include user input to a health questionnaire.

The system obtains 1420 input from the user, e.g., via the user interface presented on the client device. The system may obtain input requesting tailoring of the program. For example, the user may indicate a fear of one type of stressor. The system can record the user preference to not use that type of stressor in the program generation.

The system obtains 1430 a template for an ANS regulation program. The template may include different types of stressors, ordering of stressors, combination of stressors, parameter ranges for stressors, parameter ranges for the program, recommendations to the user, or some combination thereof.

The system generates 1440 the tailored ANS regulation program by modifying the template based on the health data and/or the input of the user. The system may generate a calibration profile of the user based on the user's health data and/or the input of the user. The calibration profile may place the user's physiological state in comparison to a population of users (e.g., among a cohort of similar background). The system modifies the template, e.g., adjusting parameters, to craft a personalized ANS regulation program for the user.

In one or more embodiments, the system may modify the ANS regulation program on-the-fly, i.e., in real-time, during performance of the ANS regulation program. The system obtains 1450 real-time biometric data from one or more health sensors. The system analyzes 1460 the real-time biometric data. The system modifies 1470 the ANS regulation program based on the analysis of the real-time biometric data.

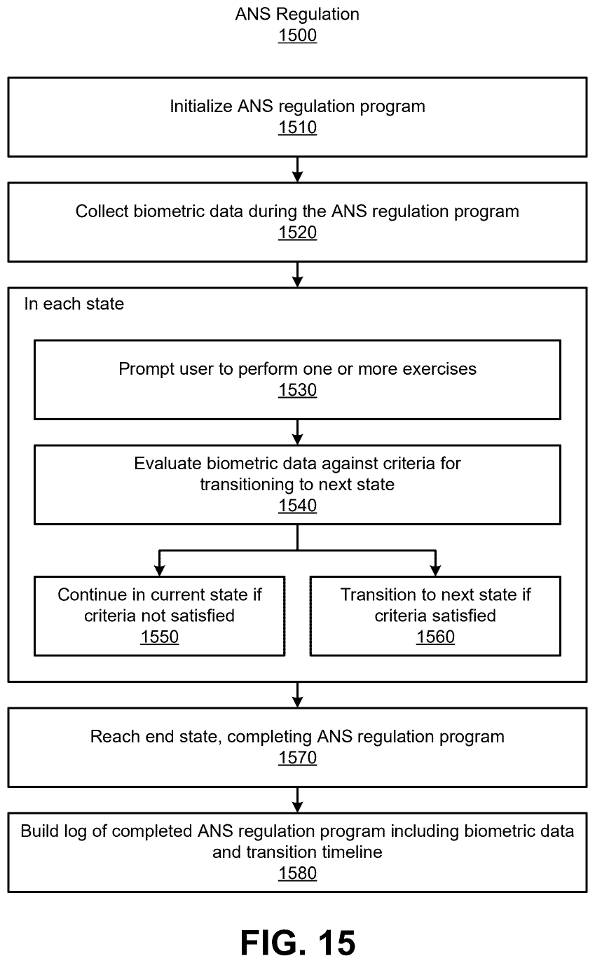

FIG. 15 illustrates a process of provision 1500 of an ANS regulation program, according to one or more embodiments. The process of provision 1500 of the ANS regulation program may be performed by a client device (e.g., the client device 100). The ANS regulation program may be tailored to an individual, e.g., by the ANS regulation system 120 or the ANS regulation system 200. The ANS regulation program may be defined by a state model, including one or more states with actions for regulation of the user's ANS and criteria for transitioning between the states. In other embodiments, the process of provision 1500 of the ANS regulation program may be performed in conjunction with a user interface. The user interface may provide prompts (e.g., visual content) that guide the user through the ANS regulation program. In other embodiments, the process may include additional, fewer, or different steps than those listed in the illustrative flowchart.

The device initializes 1510 the ANS regulation program. The device may perform the program via a user interface presented on an electronic display of the device. An initial state of the program may wait for the user to reach a stabilization point. For example, the device may measure the heart rate signal of the user and determine whether the user has stabilized based on the heart rate signal. The device can calculate a rolling HR slope, and assess whether the rolling HR slope is within a tolerance of a zero slope (e.g., within a slope's absolute value of 0.1, 0.2, 0.3, etc.). The device can calculate a rolling HR spread, and assess whether the rolling HR spread is within a threshold spread (e.g., within 2 bpm, 3 bpm, 4 bpm, 5 bpm, etc.).

The device collects 1520 biometric data during the ANS regulation program. The device may continuously collect the biometric data, e.g., via the one or more health sensors.

In each state of the state model of the ANS regulation program, the device prompts 1530 the user to perform one or more actions. The device may leverage a timing prediction model to determine start time for stressors, start time for recovery, etc.

In each state of the state model of the ANS regulation program, the device evaluates 1540 the biometric data against criteria for transitioning to the next state. The device may check the criteria of the state model to determine whether to continue in the state of transition to the next state. Each state may include a temporal limit, e.g., if a maximum limit is reached, the state model transitions. Other criteria may include achieving a target value in the biometric data, e.g., a target HR, a recovery level, etc.

In each state of the state model of the ANS regulation program, the device continues 1550 in the current state if the criteria are not satisfied. In one or more embodiments, the state model continues until the criteria are satisfied. In other embodiments, the state model may repeat the state.

In each state of the state model of the ANS regulation program, the device transitions 1560 to the next state if the criteria are satisfied. If the criteria are satisfied (which may include various logic operating on the criteria), the device may transition to the next state. The next state may include other actions (e.g., stressors).

The device reaches 1570 the end state, completing the ANS regulation program. The device may also assess whether the user is exhausted at any point throughout the program. The device may identify whether the user's ANS response to a stressor is lower than past instances of a similar stressor. If so, the device may determine the user to be exhausted, and proceed to the end state. The end state may include a meditative exercise to conclude the program.

The device builds 1580 a log of the completed ANS regulation program including the collected biometric data and the transition timeline through the state model. The device may log the transition timelines through the state model, e.g., tracking what criteria satisfied transitions between states in the state model. Such information may be leveraged by a system to analyze the user's ANS. For example, if the program concluded before completion of the states due to user exhaustion, the transition timeline can indicate such.

### Example Computing System Architecture

An example computer includes one or more processors (generally, a processor) coupled to a chipset to form a processing system. The chipset includes a memory controller hub and an input/output (I/O) controller hub. At least one memory (generally, a memory) and a graphics adapter are coupled to the memory controller hub, and a display is coupled to the graphics adapter. A storage device, a keyboard, a pointing device, a network adapter, or some combination thereof may be coupled to the I/O controller hub. Other embodiments of a computer may have different architectures.

The storage device is a non-transitory computer-readable storage medium such as a hard drive, compact disk read-only memory (CD-ROM), DVD, or a solid-state memory device. The non-transitory computer readable storage medium and/or the memory store instructions to be executed by the processor and data for use in data operations by the processor. The pointing device may be a mouse, track ball, touch-screen, or another type of pointing device, and may be used in combination with the keyboard (e.g., which may be an on-screen keyboard) to input data into the computer. The graphics adapter displays images and other information on the display. The network adapter couples the computer to one or more computer networks.

The computer system memory and/or storage medium is configured to store program code (or software). The program code comprises instructions executable by the one or more processors in the processing system. The program code corresponds to the processes described herein, e.g., with respect to FIGS. 1 through 15. Those processes may be embodied in instructions as processing steps to produce the outputs noted therein.

### Additional Considerations

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one or more embodiments, a software module is implemented with a computer program product comprising a computer-readable medium containing computer program code, which can be executed by a computer processor for performing any or all of the steps, operations, or processes described.

Embodiments of the invention may also relate to an apparatus for performing the operations herein. This apparatus may be specially constructed for the required purposes, and/or it may include a general-purpose computing device selectively activated or reconfigured by a computer program stored in the computer. Such a computer program may be stored in a non transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments of the invention may also relate to a product that is produced by a computing process described herein. Such a product may include information resulting from a computing process, where the information is stored on a non transitory, tangible computer readable storage medium and may include any embodiment of a computer program product or other data combination described herein.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

What is claimed is:

1. A computer-implemented method comprising:
initializing, via a client device, an autonomic nervous system (ANS) regulation program comprising a state model comprising a plurality of states, wherein each state includes one or more criteria for transitioning to another state of the plurality of states;
obtaining a heart rate signal, via one or more health sensors wirelessly coupled to the client device during the ANS regulation program; and
for each state of the state model:
prompting, via the client device, a user of the client device to perform one or more actions to regulate an ANS of the user,
determining whether the heart rate signal obtained during the one or more actions for the state satisfy the one or more criteria for the state, and
responsive to determining that the one or more criteria are satisfied, transitioning to another state of the plurality of states.

2. The computer-implemented method of claim 1,
wherein initializing the ANS regulation program comprises presenting, via the client device, a user interface on an electronic display of the client device,
wherein prompting the user to perform the one or more actions in each state comprises prompting the user via one or more graphical elements in the user interface presented on the electronic display of the client device.

3. The computer-implemented method of claim 1, wherein determining whether the heart rate signal obtained during the one or more actions for the state satisfy the one or more criteria for the state comprises:
determining whether the heart rate signal reaches a target heart rate for the one or more actions; and
prompting, via the client device, the user to end the one or more actions.

4. The computer-implemented method of claim 3, wherein determining whether the heart rate signal obtained during the one or more actions for the state satisfy the one or more criteria for the state comprises:
determining whether the heart rate signal reaches a recovery level, following prompting of the user to end the one or more actions.

5. The computer-implemented method of claim 1, wherein obtaining the heart rate signal comprises one or both of:
obtaining the heart rate signal from a heart rate monitor coupled to the user; and
calculating the heart rate signal from R-R intervals of an electrocardiogram signal obtained from a electrocardiogram monitor.

6. The computer-implemented method of claim 1, further comprising:
for an initial state of the state model, determining whether a physiological state of the user is stable based on the heart rate signal.

7. The computer-implemented method of claim 6, wherein determining whether the physiological state of the user is stable comprises:
calculating a slope of the heart rate signal over a past window of time; and
determining whether the slope of the heart rate signal is within a tolerance of a zero slope.

8. The computer-implemented method of claim 6, wherein determining whether the physiological state of the user is stable comprises:
calculating a spread of the heart rate signal over a past window of time as a difference between a maximum value and a minimum value in the past window of time; and
determining whether the spread of the heart rate signal is within a threshold spread.

9. The computer-implemented method of claim 1, further comprising:
for at least one state:
identifying a frequency in oscillation of the heart rate signal; and
determining a start time of an exercise based on the frequency in the oscillation of the heart rate signal to align the exercise with an incline in the heart rate signal.

10. The computer-implemented method of claim 1, further comprising:
for each state of the state model:
responsive to determining that the one or more criteria are not satisfied, continuing the state.

11. A non-transitory computer-readable storage medium storing instructions that, when executed by a computer processor, cause the computer processor to perform operations comprising:
initializing, via a client device, an autonomic nervous system (ANS) regulation program comprising a state model comprising a plurality of states, wherein each state includes one or more criteria for transitioning to another state of the plurality of states;
obtaining a heart rate signal, via one or more health sensors wirelessly coupled to the client device during the ANS regulation program; and
for each state of the state model:
prompting, via the client device, a user of the client device to perform one or more actions to regulate an ANS of the user,
determining whether the heart rate signal obtained during the one or more actions for the state satisfy the one or more criteria for the state, and
responsive to determining that the one or more criteria are satisfied, transitioning to another state of the plurality of states.

12. The non-transitory computer-readable storage medium of claim 11,
wherein initializing the ANS regulation program comprises presenting, via the client device, a user interface on an electronic display of the client device, and
wherein prompting the user to perform the one or more actions in each state comprises prompting the user via one or more graphical elements in the user interface presented on the electronic display of the client device.

13. The non-transitory computer-readable storage medium of claim 11, wherein determining whether the heart rate signal obtained during the one or more actions for the state satisfy the one or more criteria for the state comprises:
determining whether the heart rate signal reaches a target heart rate for the one or more actions; and
prompting, via the client device, the user to end the one or more actions.

14. The non-transitory computer-readable storage medium of claim 13, wherein determining whether the heart rate signal obtained during the one or more actions for the state satisfy the one or more criteria for the state comprises:
determining whether the heart rate signal reaches a recovery level, following prompting of the user to end the one or more actions.

15. The non-transitory computer-readable storage medium of claim 11, wherein obtaining the heart rate signal comprises one or both of:
obtaining the heart rate signal from a heart rate monitor coupled to the user; and
calculating the heart rate signal from R-R intervals of an electrocardiogram signal obtained from a electrocardiogram monitor.

16. The non-transitory computer-readable storage medium of claim 11, the operations further comprising:
for an initial state of the state model, determining whether a physiological state of the user is stable based on the heart rate signal.

17. The non-transitory computer-readable storage medium of claim 16, wherein determining whether the physiological state of the user is stable comprises:
calculating a slope of the heart rate signal over a past window of time; and
determining whether the slope of the heart rate signal is within a tolerance of a zero slope.

18. The non-transitory computer-readable storage medium of claim 16, wherein determining whether the physiological state of the user is stable comprises:
calculating a spread of the heart rate signal over a past window of time as a difference between a maximum value and a minimum value in the past window of time; and
determining whether the spread of the heart rate signal is within a threshold spread.

19. The non-transitory computer-readable storage medium of claim 11, the operations further comprising:
for at least one state:
identifying a frequency in oscillation of the heart rate signal; and
determining a start time of an exercise based on the frequency in the oscillation of the heart rate signal to align the exercise with an incline in the heart rate signal.

20. A system comprising:
a computer processor; and
a non-transitory computer-readable storage medium storing instructions that, when executed by the computer processor, cause the computer processor to perform operations comprising:
initializing, via a client device, an autonomic nervous system (ANS) regulation program comprising a state model comprising a plurality of states, wherein each state includes one or more criteria for transitioning to another state of the plurality of states;
obtaining a heart rate signal, via one or more health sensors wirelessly coupled to the client device during the ANS regulation program; and
for each state of the state model:
prompting, via the client device, a user of the client device to perform one or more actions to regulate an ANS of the user,
determining whether the heart rate signal obtained during the one or more actions for the state satisfy the one or more criteria for the state, and
responsive to determining that the one or more criteria are satisfied, transitioning to another state of the plurality of states.
